(19) United States
(12) Patent Application Publication
(10) Pub. No.: US 2026/0243754 A1
(43) Pub. Date: Aug. 20, 2026

(54) CONDUCTIVE AND SELECTIVELY PERMEABLE NANOREACTOR CHIP

(71) Applicant: ELECTRONIC BIOSCIENCES, INC., San Diego, CA (US)

(72) Inventors: Sean GERMAN, Salt Lake City, UT (US); Aaron REYNOLDS, Salt Lake City, CA (US); Eric Mayo PETERSON, Salt Lake City, UT (US); Eric Nathan ERVIN, Salt Lake City, UT (US)

(21) Appl. No.: 19/489,512

(22) PCT Filed: May 30, 2024
(86) PCT No.: PCT/US2024/031739

§ 371 Date: Dec. 2, 2025

Related U.S. Application Data
(60) Provisional application No. 63/470,567, filed on Jun. 2, 2023.

(51) Int. Cl. G01N 33/487

(52) U.S. Cl. CPC G01N 33/48721

(57) ABSTRACT

In alternative embodiments, provided are nanowell platforms in which the bottom of the nanowell comprises a porous layer, not an electrode, and products of manufacture comprising nanowell platforms as provided herein, and methods for making and using nanowell platforms as provided herein.

### RELATED PATENT APPLICATION

This patent application claims priority to and the benefit of U.S. Provisional Patent Application No. 63/470,567, filed on Jun. 2, 2023. The entire content of the foregoing patent application, including all text and drawings, is herein incorporated by reference for all purposes.

### TECHNICAL FIELD

This invention generally relates to nanotechnology and biochips. In alternative embodiments, provided are nanowell platforms in which the bottom of the nanowell comprises a porous layer, not an electrode, and products of manufacture comprising nanowell platforms as provided herein, and methods for making and using nanowell platforms as provided herein.

### BACKGROUND

Current flowing through through-hole, microwells, nanowells, recessed electrodes, nanopore electrodes, and electrode chips, as well as entities/structure contained in, on, or over these structures, such as planar lipid bilayers, membranes and associated nanopore/ion channels is an ionic current as charged electrolytes are driven through and across them via an applied potential. Electrodes on opposite sides of the structure (typically a working electrode at the bottom of the well and a large area reference electrode outside of the well) serve not only to apply the potential difference that drives the current, but also play an essential role in converting ionic current to an electric one via a built-in redox system. For example, one of the most commonly employed reference systems is the Ag/AgCl electrode where for every ion pair passing through the pore (K⁺ going trans to cis and Cl⁻ moving cis to trans) an electron transfer event must occur at each reference electrode Ag₍ₛ₎→Ag⁺ to balance the Cl⁻ and vice versa at the opposite electrode to maintain electroneutrality. Without these electron transfer events at the reference electrode, the current will rapidly shut off as the uncompensated ions generate a potential to cancel out the applied voltage.

Decreasing the size of a reaction volume is often desirable in order to multiplex a measurement, to study single molecules, or to increase the effective concentration and therefore reaction rates of entities interacting within the reaction volume. Integrating a miniaturized reference electrode within these reduced volumes presents a challenge in that it must be comprised of sufficient redox material (contain enough charge capacity) to drive current for an appreciable amount of time.

Additionally, long-term stability of the electrode is an important consideration as various chemical processes (e.g., leaching of entrapped redox couples and dissolution of silver chloride via soluble chloride complexes) can become significant for small surface area electrodes leading to charge capacity loss.

### SUMMARY

In alternative embodiments, provided are nanowell chips, comprising: a substrate comprising a substrate posterior surface, a substrate anterior surface and a substrate bore; the substrate bore comprising a posterior bore opening disposed at the substrate posterior surface and an anterior bore opening disposed at the substrate anterior surface; an electrode layer joined to a portion of the substrate anterior surface, the electrode layer comprising an anterior surface and a posterior surface comprising a portion disposed at the anterior bore opening; an insulator layer disposed on the electrode layer anterior surface and the substrate anterior surface, the insulator layer comprising an insulator layer anterior surface and an insulator layer posterior surface; a porous layer disposed on the electrode layer portion, the porous layer comprising a porous layer anterior surface joined to the electrode layer portion and a porous layer posterior surface disposed in the substrate bore; and a nanowell disposed in the insulator layer and in the electrode layer, the nanowell comprising a nanowell anterior opening disposed at the insulator layer anterior surface and a nanowell posterior terminus disposed at the porous layer anterior surface, wherein the nanowell is aligned with the anterior bore opening of the substrate. In alternative embodiments, provided are products of manufacture comprising a nanowell chip as provided herein.

The details of one or more exemplary embodiments of the invention are set forth in the accompanying drawings and the description below. This Summary section is not limiting and other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The drawings set forth herein are illustrative of exemplary embodiments provided herein and are not meant to limit the scope of the invention as encompassed by the claims.

FIG. 1A schematically illustrates an exploded view of stacked layers and films comprising an exemplary nanowell chip assembly as provided herein (not to scale), FIG. 1B is a non-exploded cross-section view thereof (not to scale), and FIG. 1C is a non-exploded cross-section view showing assembly components of FIG. 1B connected by a clamp (not to scale).

FIG. 2A to FIG. 2F schematically illustrate different embodiments of exemplary nanowell structures as provided herein (not to scale).

FIG. 3A to FIG. 3D schematically illustrate different embodiments of exemplary nanowell chip, working electrode and reference electrode assemblies (not to scale).

FIG. 4 schematically illustrates general fabrication scheme for an exemplary nanowell chip as provided herein.

FIG. 5A schematically illustrates an exemplary nanowell chip assembly (not to scale), and FIG. 5B is an enlarged view of the portion designated by a broken line box in FIG. 5A.

FIG. 6A to FIG. 6G schematically illustrate an exemplary nanowell chip assembly as provided herein. FIG. 6A schematically illustrates stacked layers and films comprising the assembly (not to scale). FIG. 6B schematically illustrates a complete prototype nanowell system. FIG. 6C illustrates an individual two-plex chip, FIG. 6D illustrates a gold lead aligned over an etched glass hemisphere, and FIG. 6E illustrates a nanowell etched in parylene, for an exemplary nanowell chip. FIG. 6F shows a brightfield image from the bottom of the nanowell. FIG. 6G shows a darkfield image of same area in FIG. 6F showing a light scattering silica aggregate floating on top of the mesoporous silica film that spans the bottom of the nanowell.

FIG. 7A and FIG. 7B schematically illustrates electrical characterization of mesoporous silica. FIG. 7A shows i-V curves recorded with 1M KCl and FIG. 7B shows conductance, recorded from an open aperture (dashes), mesoporous silica with the CTAB template extracted (dots), a single alpha-hemolysin pore (line shown in FIG. 7A and not in FIG. 7B), and mesoporous silica with the CTAB template still in place (dash-dot).

Like reference symbols in the various drawings indicate like elements.

### DETAILED DESCRIPTION

In alternative embodiments, provided are nanowell platforms in which the bottom of the nanowell comprises a porous layer, not an electrode, and products of manufacture comprising nanowell platforms as provided herein, and methods for making and using nanowell platforms as provided herein. In alternative embodiments, provided are nanowell platforms in which the bottom of the nanowell is capped by a porous layer, not an electrode.

Through-hole, microwells, nanowells, recessed electrodes, nanopore electrodes, and electrode chips are miniaturized structures created by developing micro and nanoscale wells or holes in various materials, such as polymers, insulators, photoresists, films, or substrates. Typically, a recessed microelectrode is placed within these structures to facilitate recording of electrical properties. These technologies have a broad range of applications in various industries, including the medical field for studying cell signaling and drug discovery, developing biosensors for disease detection, and for environmental monitoring, such as detecting contaminants in water sources. Additionally, these technologies can be used for ion channel and nanopore measurements, polymer sequencing applications, and as nanoreactors. The miniaturization of these technologies enables large arrays, reduces test volumes for lowering reagent costs, and increases effective concentrations of translocating species in nanoreactor applications.

Provided herein is a nanowell platform, in which the bottom of the nanowell contains, comprises or is capped by a porous layer, not an electrode. These devices have similar utility to devices that do contain electrodes at their bottom, but overcome challenges with the latter, such as electrode longevity, ion accumulation/depletion within their confined volume, for example.

In applications where femtoliter or smaller volumes are desired, simply adding an electrode to the bottom of the well typically cannot provide current for more than a few seconds. To enable sustained electrical measurements, provided herein is a device containing a nanoscale confined volume in an anterior position relative to, and in electrochemical communication with, a separated electrode (for example, a separated macroscale electrode), connected via a porous layer in a posterior position relative to the nanoscale confined volume. A nanoscale confined volume also is referred to as a nanowell. Electrochemical communication also is referred to as electroconductive association, and a confined volume typically is in electroconductive association with a separated electrode counterpart.

A nanoscale confined volume sometimes is in a chip of a device, also referred to as a nanowell chip. A nanoscale confined volume sometimes contains a volume of about 10,000 nanoliters (nL) or less, 1,000 nL or less, or about 500 nL or less, or about 100 nL or less, or about 10 nL or less, or about 1 nL or less, or about 100 picoliters (pL) or less, about 10 pL or less, or about 1 pL or less, or about 100 femtoliters (fL) or less, about 10 fL or less, or about 1 fL or less, or about 100 attoliters (aL) or less, or about 10 aL or less, or about 1 aL or less, or about 100 zeptoliters (zL) or less, or about 10 zL or less. A nanoscale confined volume can be any suitable geometry (for example, cube, cuboid, cylinder, pyramidal, cylindroid), and can be structured with angled side walls or have scalloped/ridged edges that can facilitate engineering a lipid bilayer annulus/contact angle. A nanoscale confined volume sometimes is an average, mean, median or nominal volume. A device or nanowell chip of a device can contain one nanowell or can contain an array of nanowells. An array of nanowells can include about 2 to about 100,000 nanowells, or about 10 to about 10,000 nanowells, about 2 to about 1100 nanowells, or about 2, 4, 8, 16, 32, 64, 100, 128, 256, 512, or 1024 nanowells.

A separated electrode in electrochemical communication with a particular nanowell or plurality of nanowells is referred to as a working electrode. A working electrode typically is in use as an electrode when a nanowell chip is utilized to analyze an analyte. While a nanowell chip optionally can include an electrode layer disposed between a substrate and an insulator layer in certain specific implementations, such an electrode or metallic layer typically is utilized in one or more aspects of manufacturing the nanowell chip, typically is not utilized as an electrode when using the chip to analyze an analyte, and typically is not considered a working electrode. An electrode layer in a chip can be a metallic layer.

A separated working electrode in electroconductive association and/or fluidic association with a particular nanowell or a plurality of nanowells typically is separated by a distance from the floor of the associated nanowell(s), and can be separated by a distance from a porous layer of the associated nanowell(s). A nanowell floor typically is a posterior surface within a nanowell interior. A nanowell floor often is an anterior surface of a porous layer exposed to a nanowell interior in a nanowell chip described herein. A working electrode often is disposed in a trans position relative to a nanowell anterior opening (below demarcation 409 for example).

In alternative embodiments, a minimum distance between an anterior surface of a working electrode and a floor of a corresponding nanowell (a minimum distance between an anterior surface of a working electrode and a posterior terminus of a nanowell in electroconductive association with the electrode, for example) can be about 1 to about 5000 micrometers, or about 1 to about 50 micrometers, or about 25 to about 250 micrometers, or about 500 to about 2000 micrometers (a minimum distance along axial direction 405 for example). In alternative embodiments, each working electrode includes a width of about 10 micrometers to about 1000 or about 10 micrometers to about 150 micrometers or about 100 micrometers to about 500 micrometers or about 250 micrometers to about 10000 micrometers (along radial axis 407 for example). In alternative embodiments each of the nanowells comprises a width of about 50 to about 5000 nanometers, or about 50 nanometers to about 250 nanometers, or about 100 nanometers to about 1000 nanometers, or about 1000 nanometers to about 5000 nanometers, or about 5000 nanometers to about 100000 nanometers (along radial axis 407 for example).

In alternative embodiments, each nanowell comprises a width of about 500 nanometers to about 1000 nanometers or about 1000 nanometers to about 5000 nanometers (along radial axis 407 for example). In alternative embodiments, an associated working electrode comprises a width of about 10 micrometers to about 100 micrometers (along radial axis 407 for example). In alternative embodiments, a ratio between a working electrode width to an associated nanowell width (working electrode width divided by width of associated nanowell) is about 2 to about 200,000, or about 2 to about 20,000, or about 2 to about 2,000, or about 2 to about 200, or about 5 to about 100, or about 10 to about 80, or about 10 to about 30 or about 55 to about 75. The width of a nanowell can be at the nanowell floor or posterior terminus. A working electrode and an associated nanowell of a nanowell chip (also referred to as a confined volume) typically are in electroconductive association (described herein).

For implementations in which there are two or more confined volumes in a system (for example, two or more nanowells in a nanowell chip) and two or more separated working electrodes (for example, in an electrode chip), each individual nanowell typically is in electroconductive association with a single separated working electrode and not with other separated working electrodes. Each confined volume can be in electroconductive association with a separated working electrode via fluid that often contains a supporting electrolyte. Each confined volume can be in electroconductive association with a single separated working electrode by partitioning fluid in contact with one confined volume and its corresponding separated working electrode from other confined volumes and their corresponding separated electrodes. Partitioning can be facilitated by a partition member in a system that fluidically separates (fluidically partitions) each confined volume and corresponding separated working electrode from each other confined volume and corresponding separated working electrode. A partition member can be a gasket containing an orifice in a plurality of orifices, where one orifice is in fluid association with one confined volume and one corresponding separated working electrode and is not in fluid association with another confined volume and its corresponding separated electrode, for example. A partition member can be a fluidic channel in a plurality of fluidic channels, where one fluidic channel is in fluid association with one confined volume and one corresponding separated electrode and is not in fluid association with another confined volume and its corresponding separated electrode, for example.

In alternative embodiments, a system or assembly includes a reference electrode, separate from each working electrode. A system or assembly containing multiple nanowells can include one reference electrode or a plurality of reference electrodes. For implementations in which a working electrode is in a posterior or trans position relative to a nanowell anterior opening, a reference electrode can be positioned in an anterior or cis position relative to a nanowell anterior opening or can be positioned in bulk solution.

In alternative embodiments, a nanowell chip often includes no electrode disposed at a nanowell floor (no working electrode disposed at a nanowell floor, for example), and/or often includes no working or reference electrode disposed at an optional electrode layer posterior surface (no working electrode disposed at an optional electrode layer posterior surface, for example), and/or often includes no working electrode or reference disposed on a porous layer posterior surface or anterior surface.

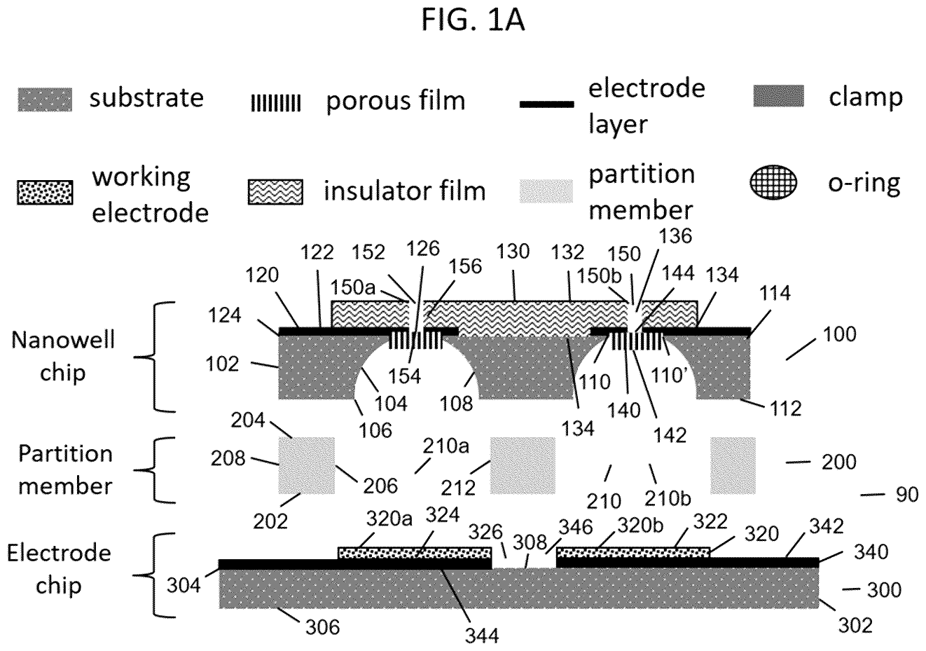

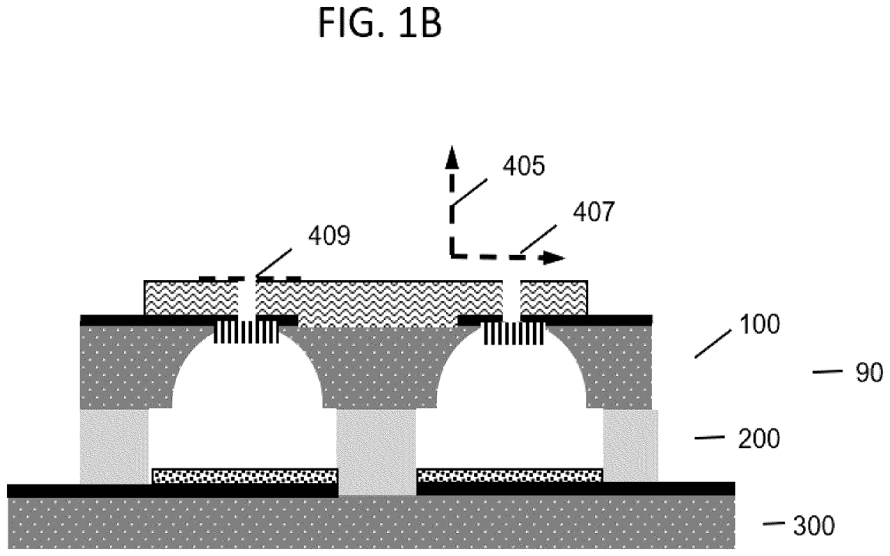

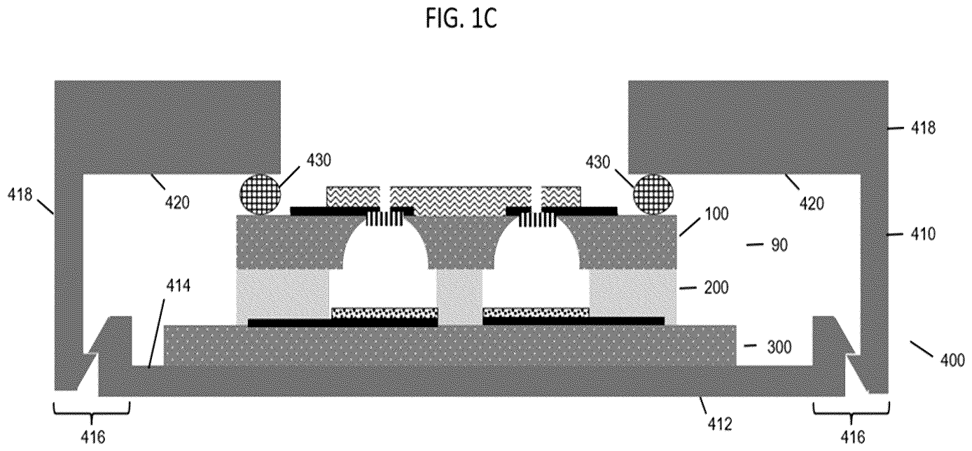

In alternative embodiments, a separated electrode in association with a nanowell of a nanowell chip typically includes a width greater than the width of an associated nanowell counterpart and can be a macroscale electrode. A width typically is a measurement parallel to radial direction 407 illustrated in FIG. 1B. A separated electrode sometimes includes a width of about 10 micrometers (μm) or greater, or about 100 μm or greater, or about 1,000 mm or greater. A separated electrode is of any suitable geometry (for example, quadrilateral, square, rectangle, triangular, circle, oval, ovoid), an electrode width sometimes is an average, mean, median or nominal width, and sometimes an electrode width is a diameter. A separated electrode sometimes is disposed in a posterior position relative to a nanowell anterior opening, and often is a working electrode for the nanowell counterpart.

In alternative embodiments, a porous layer, which also is referred to herein as a permeable layer and a porous film, contains an array of vertical (axial) pores that sometimes are negatively charged pores. The width of the pores of a porous layer sometimes is about 0.1 nanometers (nm) to about 10 nm, or about 0.5 nm to about 5 nm, or about 1 nm to about 2 nm, or about 1.5 nm or about 1.6 nm, or about 0.1, 0.2, 0.3, 0.4, 0.5, 0.6, 0.7, 0.8, 0.9, 1, 2, 3, 4, 5, 6, 7, 8, 9 or 10 nm. The center to center distance of pores in the regularly or randomly distributed array can be about 2 nm to about 50000 nm, about 4 nm to about 1000 nm, or about 3, 5, 10, 100, 500, or 1000 nm. A pore width sometimes is an average, mean, median or nominal width, and sometimes a pore width is a diameter.

A porous layer sometimes includes a mesoporous silica layer, and/or can include a different material as described herein. A porous layer can serve as a low resistance “frit” that allows ion transport for driving current, while still enclosing the internal micro or nanowell volume via size exclusion and/or charge repulsion preventing transport of non-supporting electrolyte molecules down to the larger volume. A permeable layer includes a width that often is greater than the width of the nanowell with which it is associated (for example, the width of the permeable layer often is greater than the width of the nanowell posterior terminus). The width of the permeable layer and the nanowell width typically are in radial direction 407. A permeable layer often is not disposed in a nanowell in a position anterior to a nanowell posterior terminus. An anterior surface of a permeable layer often is disposed at a nanowell posterior terminus, and can serve as a nanowell posterior terminus or nanowell floor. A permeable layer may be of any suitable shape and sometimes is quadrilateral, square, rectangular, triangular, oval, ovoid or circular, and sometimes a width is a diameter. A device containing a nanoscale reaction volume with a confining, selectively permeable layer (for example, a mesoporous silica barrier membrane) allows use of large, highly stable electrodes in both the trans and cis volumes for high-bandwidth, long-lived recording. Individual nanoscale confined volumes arrayed on a chip may be addressed separately when isolated fluidically via a gasket or microfluidics on the trans side and placed on a second chip with coplanar, patterned electrodes with leads and contact pads to interface with the control electronics.

In alternative embodiments, a nanowell chip includes a substrate containing one or more bores. A nanowell chip containing one or more substrate bores sometimes includes an array of substrate bores. Each bore often includes a bore anterior opening disposed at a substrate anterior surface and often includes a bore posterior opening disposed at a substrate posterior surface. Each bore contains a sidewall of a suitable geometry to facilitate nanowell chip fabrication and analysis of an analyte, and sometimes includes a curved surface and/or angled surface, and sometimes a substrate bore is defined at least in part by a hemisphere, hemispheroid, frustrum (for example, a right frustrum, a truncated cone, a truncated pyramid), cylinder or cylindroid volume, for example. Each nanowell of a nanowell chip typically is associated with one substrate bore.

A nanowell chip sometimes includes an optional electrode layer, which when present often is disposed on the substrate anterior surface and often is patterned. A nanowell often is disposed in an insulator layer in a nanowell chip. An insulator layer sometimes is disposed on an anterior surface of an optional electrode or metallic layer, for implementations in which a nanowell chip includes an electrode layer. An insulator layer sometimes is disposed on the substrate anterior surface for implementations in which a nanowell chip includes no optional electrode layer.

Each nanowell of a nanowell chip typically is associated with a porous layer. A porous layer of a nanowell chip sometimes is continuous, or sometimes is discontinuous where a porous layer portion is associated with one nanowell and a separated porous layer portion is associated with another nanowell. A porous layer of a nanowell chip sometimes is disposed on a posterior surface of an electrode layer, and sometimes is disposed on a posterior surface of an electrode layer and on a posterior surface of a substrate, for implementations in which a nanowell chip includes an optional electrode layer. A porous layer of a nanowell chip sometimes is disposed entirely on a posterior surface of an insulator layer, for implementations in which a nanowell chip includes no optional electrode layer. A porous layer or portion thereof often is disposed within a substrate bore, and a porous layer perimeter sometimes is defined or is in part defined by the substrate bore anterior opening. In certain implementations, a substrate is a porous material and there is no separate porous layer. For example, an insulator layer containing one or more nanowells can be disposed on the anterior surface of a porous substrate, or an insulator layer containing one or more nanowells can be disposed on the anterior surface of an optional electrode layer that is disposed on the anterior surface of a porous substrate.

In alternative embodiments, the utilization of the nanoscale confined volume with a porous bottom and a separated electrode (for example, an electrode having a width greater than 100 mm (in radial direction 407 for example)) underneath differs from standard recessed electrode, where the electrode resides at the bottom of the well cavity within a given substrate, with either a Faradaic/electrochemical electrode (e.g., Ag/AgCl using a DC bias) or non-Faradaic electrode (e.g., Au using an AC bias). For example, a 1 μm diameter Ag/AgCl electrode, as would be the case if the electrode were placed at the bottom of the nanoscale confined volume, has a limited lifetime (on the order of a few seconds), even when utilizing lifetime extending processes such as voltage toggling and conductive polymer coatings. Capacitively coupled micro-Au electrodes, on the other hand, suffer from limited voltage control and significantly unstable current baselines. Thus, the nanoscale confined volume structure in combination with a porous layer described here can overcome stability/control limitations of traditional nanoelectrode systems. The term “Au” typically refers to gold and “Ag” typically refers to silver.

In alternative embodiments, the nanoscale confined volume can serve to increase the effective concentration of analytes. For example, after translocating a single molecule from a bulk solution containing only femtomolar concentrations, the effective concentration of that single molecule within a 500 nanometer wide by 500 nanometer deep confined volume becomes approximately 17 nanomolar. Increased effective concentrations can increase rates of interactions between analytes and other species associated with the nanowell such as nanopore readers and immobilized enzymes, for example.

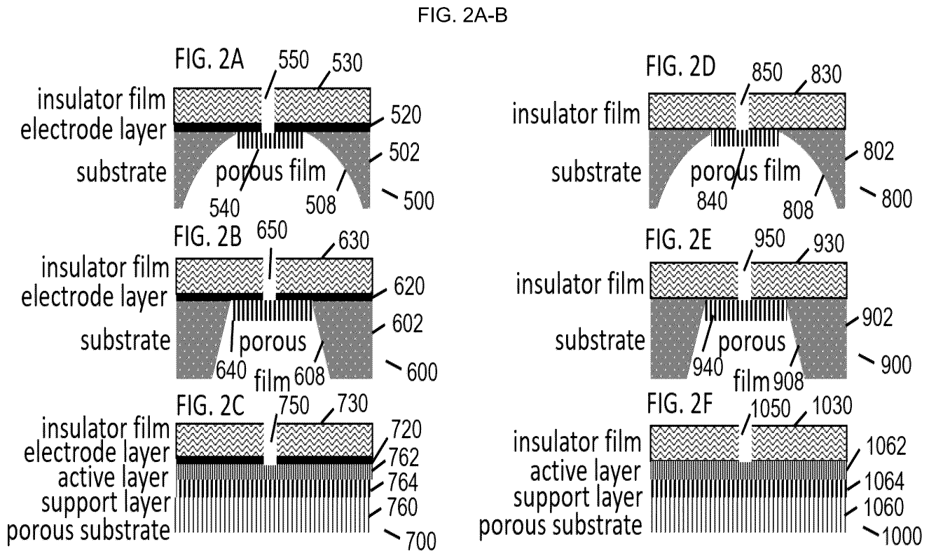

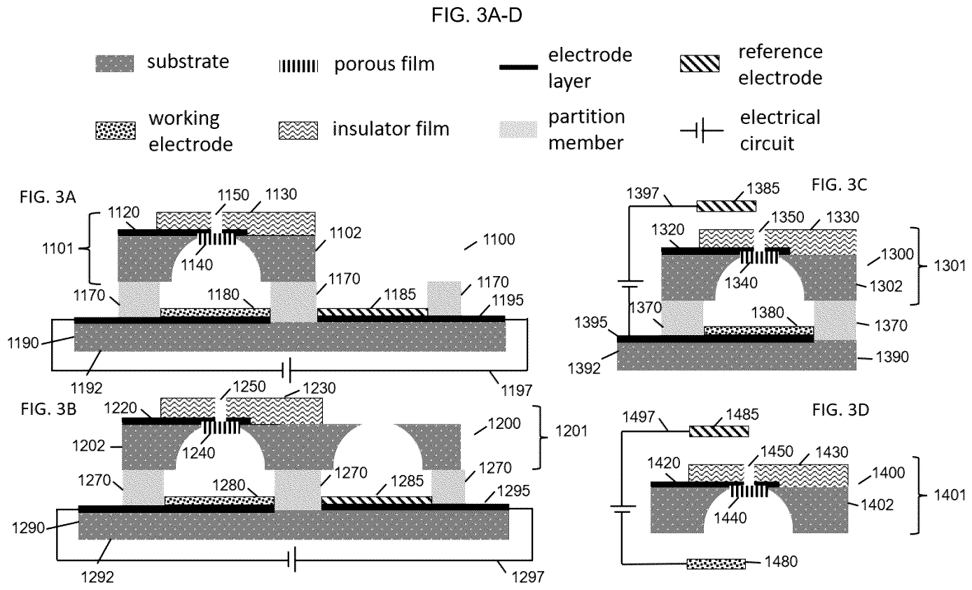

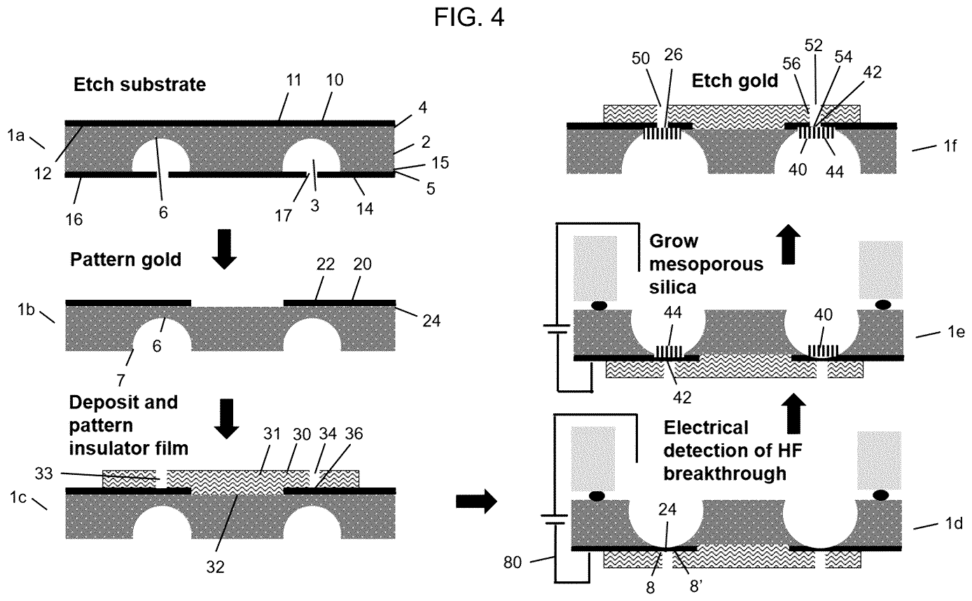

Methodology described herein can be utilized to fabricate a chip containing at least one nanoscale confined volume able to support planar lipid bilayers/membranes and channel forming entity insertion, immobilization of enzymes, catalysts, or other molecular machinery within the confined volume, and recording of ionic current across its selectively permeable defining membrane/bottom. Nanowell chips can be seated onto gaskets/microfluidics that define electrically isolated connections to macroelectrodes and contacts on a separate electrode chip. The drawings illustrate certain implementations of the technology and are not limiting. For clarity and ease of illustration, the drawings are not made to scale and, in some instances, various aspects may be shown exaggerated or enlarged to facilitate an understanding of particular implementations. FIG. 1A illustrates an exploded cross-section view of a nanowell chip assembly, and FIG. 1B illustrates an assembled cross-section view of the assembly. FIG. 2A, FIG. 2B, FIG. 2D and FIG. 2E illustrate different substrate bore configurations, and FIG. 2C and FIG. 2F illustrate a porous substrate serving as a combined substrate and porous layer. FIG. 3A to FIG. 3D illustrate different electrode configurations. FIG. 4 illustrates steps of a non-limiting example of a process for manufacturing a non-limiting example of a nanowell chip.

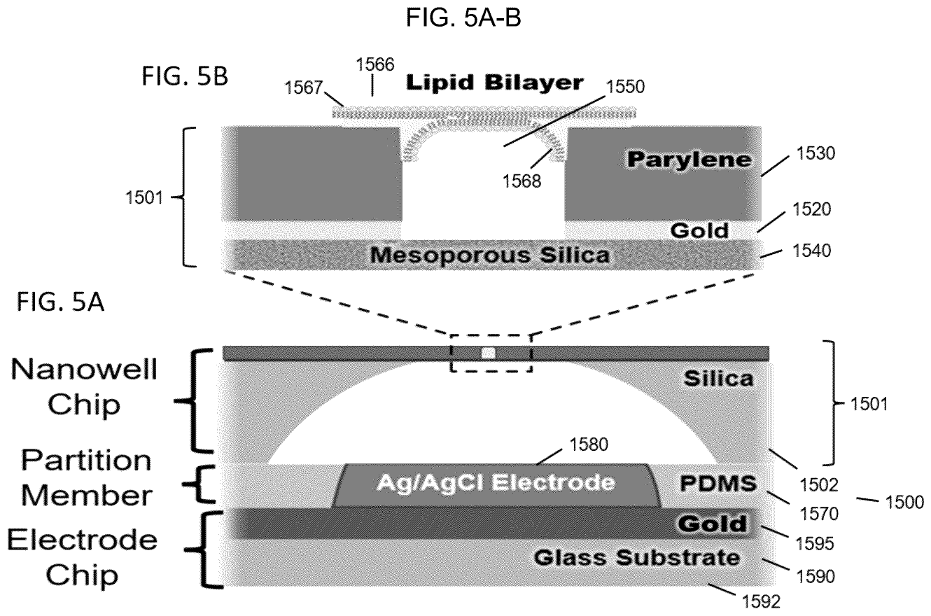

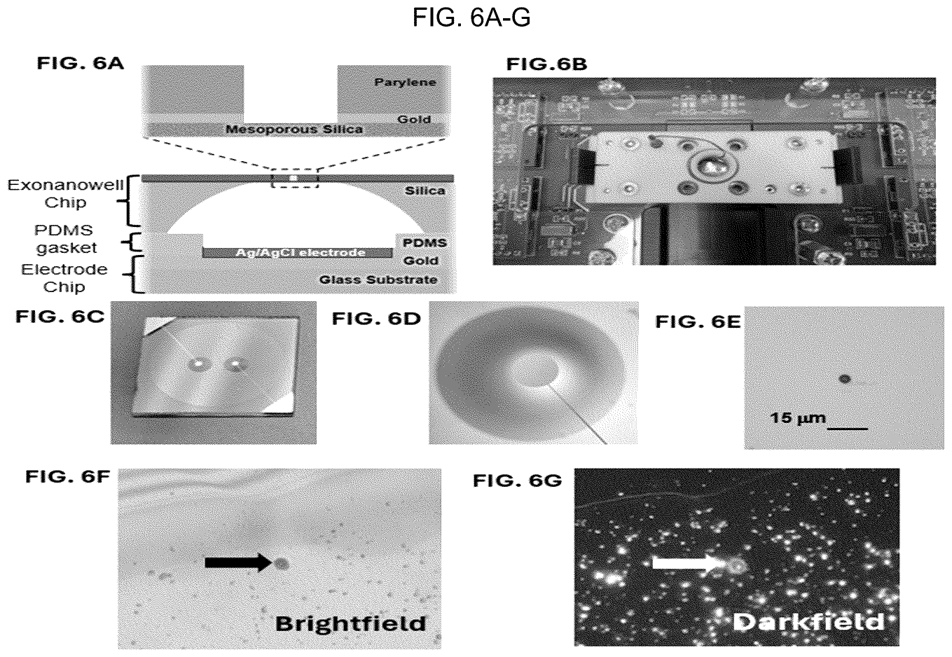

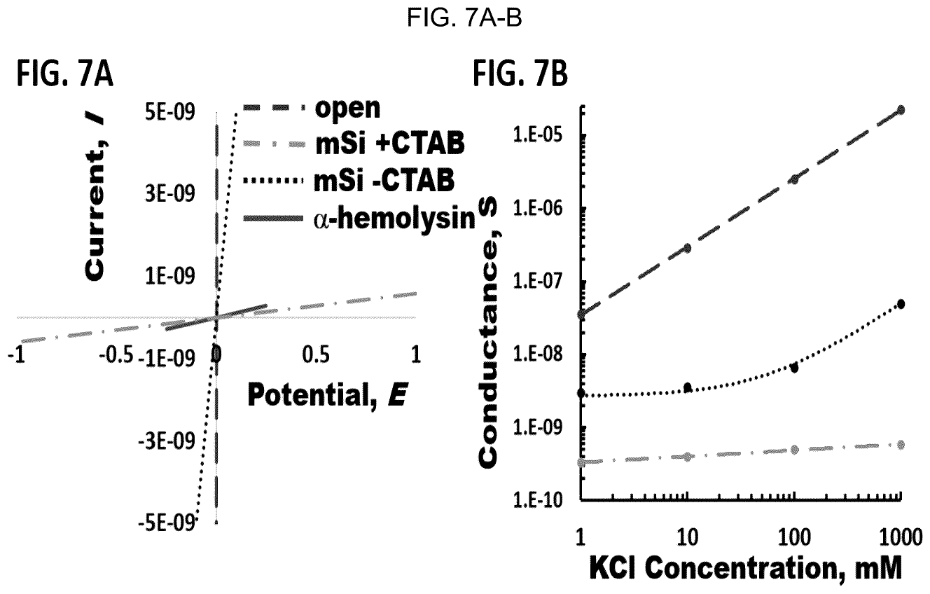

FIG. 5A and FIG. 5B illustrate a non-limiting example of a planar lipid bilayer or membrane over the nanowell chip. FIG. 5B is an enlarged view of the portion of FIG. 5A bounded by a broken-line rectangle and schematically illustrates a lipid bilayer membrane over and spanning the anterior opening of the nanowell in the insulator layer. This lipid bilayer can also be any suitable membrane material. The gold layer can serve as an electrode layer or metallic layer and gold can be substituted by another electrode material and/or metallic material (also applicable to FIG. 6A). FIG. 6A to FIG. 6G illustrate aspects of a non-limiting example of a nanowell chip assembly, with FIG. 6A illustrating a cross section view of the assembly; FIG. 6B illustrating a top view of the assembly; FIG. 6C, FIG. 6D, and FIG. 6E illustrating a top view of a nanowell chip portion; and FIG. 6F and FIG. 6G illustrating a bottom view of a nanowell chip portion. FIG. 7A and FIG. 7B illustrate aspects of electrical characterization of the mesoporous silica layer of the nanowell chip assembly illustrated in FIG. 6A to FIG. 6G. The following table describes elements of devices illustrated in the drawings.

| Callout | Element |
|---|---|
| nanowell chip precursor | 1a, 1b, 2c, 1d, 1e |
| nanowell chip | 1f |
| substrate | 2 |
| substrate bore | 3 |
| substrate anterior surface | 4 |
| substrate posterior surface | 5 |
| bore anterior terminus | 6 |
| posterior bore opening | 7 |
| anterior bore opening | 8, 8′ |
| anterior electrode layer | 10 |
| anterior electrode layer anterior surface | 11 |
| anterior electrode layer posterior surface | 12 |
| posterior electrode layer | 14 |
| posterior electrode layer anterior surface | 15 |
| posterior electrode layer posterior surface | 16 |
| posterior electrode layer orifice | 17 |
| anterior electrode layer portion | 20 |
| anterior electrode layer portion anterior surface | 22 |
| anterior electrode layer portion posterior surface | 24 |
| etched anterior electrode layer portion | 26 |
| insulator layer | 30 |
| insulator layer anterior surface | 31 |
| insulator layer posterior surface | 32 |
| insulator layer hole | 33 |
| insulator layer hole anterior opening | 34 |
| insulator layer hole posterior terminus | 36 |
| porous layer | 40 |
| porous layer anterior surface | 42 |
| porous layer posterior surface | 44 |
| nanowell | 50 |
| nanowell anterior opening | 52 |
| nanowell posterior terminus | 54 |
| nanowell sidewall | 56 |
| porous nanowell device assembly | 90 |
| nanowell chip | 100 |
| substrate | 102 |
| substrate bore | 104 |
| posterior bore opening | 106 |
| bore interior wall | 108 |
| anterior bore opening | 110, 110′ |
| substrate posterior surface | 112 |
| substrate anterior surface | 114 |
| electrode layer | 120 |
| electrode layer anterior surface | 122 |
| electrode layer posterior surface | 124 |
| electrode layer orifice | 126 |
| insulator layer (also referred to as “insulator film”) | 130 |
| insulator layer anterior surface | 132 |
| insulator layer posterior surface | 134 |
| insulator layer orifice | 136 |
| porous layer (also referred to as “porous film”) | 140 |
| porous layer posterior surface | 142 |
| porous layer anterior surface | 144 |
| nanowell | 150, 150a, 150b |
| nanowell anterior opening | 152 |
| nanowell posterior terminus | 154 |
| nanowell sidewall | 156 |
| partition member | 200 |
| partition member posterior surface | 202 |
| partition member anterior surface | 204 |
| partition member interior wall | 206 |
| partition member exterior wall | 208 |
| partition member cavity | 210, 210a, 210b |
| partition member portion | 212 |
| electrode chip | 300 |
| substrate | 302 |
| substrate anterior surface | 304 |
| substrate posterior surface | 306 |
| depression | 308 |
| separated electrode (also referred to as working | 320, 320a, 320b |
| electrode) | |
| separated electrode anterior surface | 322 |
| separated electrode posterior surface | 324 |
| separated electrode orifice | 326 |
| intermediary layer | 340 |
| intermediary layer anterior surface | 342 |
| intermediary layer posterior surface | 344 |
| intermediary layer orifice | 346 |
| clamp assembly | 400 |
| axial direction | 405 |
| radial direction | 407 |
| demarcation for cis and trans orientations | 409 |
| clamp | 410 |
| first clamp member | 412 |
| clamping surface of first clamp member | 414 |
| hinge member | 416 |
| second clamp member | 418 |
| clamping surface of second clamp member | 420 |
| deformable clamp intermediate | 430 |
| nanowell chip | 500 |
| substrate | 502 |
| electrode layer | 520 |
| insulator layer | 530 |
| porous layer | 540 |
| nanowell | 550 |
| nanowell chip | 600 |
| substrate | 602 |
| electrode layer | 620 |
| insulator layer | 630 |
| porous layer | 640 |
| nanowell | 650 |
| nanowell chip | 700 |
| insulator layer | 730 |
| nanowell | 750 |
| porous substrate | 760 |
| active layer | 762 |
| support layer | 764 |
| nanowell chip | 800 |
| substrate | 802 |
| electrode layer | 820 |
| insulator layer | 830 |
| porous layer | 840 |
| nanowell | 850 |
| nanowell chip | 900 |
| substrate | 902 |
| electrode layer | 920 |
| insulator layer | 930 |
| porous layer | 940 |
| nanowell | 950 |
| nanowell chip | 1000 |
| insulator layer | 1030 |
| nanowell | 1050 |
| porous substrate | 1060 |
| active layer | 1062 |
| support layer | 1064 |
| nanowell chip assembly | 1100 |
| nanowell chip | 1101 |
| substrate | 1102 |
| electrode layer | 1120 |
| insulator layer | 1130 |
| porous layer | 1140 |
| nanowell | 1150 |
| partition member | 1170 |
| working electrode | 1180 |
| reference electrode | 1185 |
| electrode chip | 1190 |
| electrode chip substrate | 1192 |
| electrode chip intermediary layer | 1195 |
| electrical circuit | 1197 |
| nanowell chip assembly | 1200 |
| nanowell chip | 1201 |
| substrate | 1202 |
| electrode layer | 1220 |
| insulator layer | 1230 |
| porous layer | 1240 |
| nanowell | 1250 |
| partition member | 1270 |
| working electrode | 1280 |
| reference electrode | 1285 |
| electrode chip | 1290 |
| electrode chip substrate | 1292 |
| electrode chip intermediary layer | 1295 |
| electrical circuit | 1297 |
| nanowell chip assembly | 1300 |
| nanowell chip | 1301 |
| substrate | 1302 |
| electrode layer | 1320 |
| insulator layer | 1330 |
| porous layer | 1340 |
| nanowell | 1350 |
| partition member | 1370 |
| working electrode | 1380 |
| reference electrode | 1385 |
| electrode chip | 1390 |
| electrode chip substrate | 1392 |
| electrode chip intermediary layer | 1395 |
| electrical circuit | 1397 |
| nanowell chip assembly | 1400 |
| nanowell chip | 1401 |
| substrate | 1402 |
| electrode layer | 1420 |
| insulator layer | 1430 |
| porous layer | 1140 |
| nanowell | 1450 |
| working electrode | 1480 |
| reference electrode | 1485 |
| electrical circuit | 1497 |
| nanowell chip assembly | 1500 |
| nanowell chip | 1501 |
| substrate | 1502 |
| electrode layer | 1520 |
| insulator layer | 1530 |
| porous layer | 1540 |
| nanowell | 1550 |
| lipid bilayer membrane | 1566 |
| first lipid layer | 1567 |
| second lipid layer | 1568 |
| partition member | 1570 |
| working electrode | 1580 |
| electrode chip | 1590 |
| electrode chip substrate | 1592 |
| electrode chip intermediary layer | 1595 |

Generally, a component disposed in a top position, when a system or device is oriented as the specific system implementation illustrated of FIG. 1A or FIG. 1B, typically is characterized as an anterior component. A component disposed in a bottom orientation, when the system or device is oriented as the specific system implementation illustrated in FIG. 1A or FIG. 1B, typically is characterized as a posterior component. A component or feature disposed in an anterior position relative to a nanowell anterior opening, above virtual demarcation 409 shown in FIG. 1B, typically is characterized as being in a cis position, and a component or feature disposed in a posterior position relative to a nanowell anterior opening, below virtual demarcation 409 shown in FIG. 1B, typically is characterized as being in a trans position.

### Exemplary Devices

The general structures of specific implementations of nanowell chips and electrode chips are depicted in FIG. 1A and FIG. 1B. FIG. 1A (exploded view) and FIG. 1B (non-exploded assembly view) show a cross-section view of an exemplary porous nanowell device assembly 90, which includes nanowell chip 100, electrode chip 300 and intermediary partition member 200. A posterior surface portion of nanowell chip 100 is in contact with an anterior surface of partition member 200, and an anterior surface portion of electrode chip 300 is in contact with a posterior surface of partition member 200. Nanowell chip 100, electrode chip 300 and partition member 200 each can be provided separately or two or all three can be provided together (in a kit or article or manufacture for example).

Exemplary nanowell chip 100 includes substrate 102 having substrate posterior surface 112 and substrate anterior surface 114 and containing a plurality of substrate bore 104. Each substrate bore 104 includes posterior bore opening 106, bore interior wall 108 and anterior bore opening 110, 110′. While each substrate bore 104 in nanowell chip 100 is a dome with posterior bore opening 106 having a width (along radial direction 407) greater than the width of anterior bore opening 110, and with bore interior wall 108 curved from posterior bore opening 106 to anterior bore opening 110, the bore can have any suitable geometry for containing porous layer 140 and disposing an electrode in electroconductive association with a corresponding nanowell 150. A substrate bore can include an angled, linear sidewall, such as depicted in FIG. 2B (sidewall 608) or FIG. 2E (sidewall 908) for example.

Exemplary nanowell chip 100 includes optional electrode layer 120 that includes electrode layer anterior surface 122 and electrode layer posterior surface 124. Optional electrode layer 120 can be a metallic layer. The electrode layer posterior surface 124 of optional electrode layer 120 is disposed on, and optionally is bonded or adhered to, substrate anterior surface 114. Optional electrode layer 120 of nanowell chip 100 can be patterned and can include a plurality of electrode layer orifice 126. An electrode layer orifice 126 can have any suitable geometry (as described for a nanowell herein) and can be circular for example. An electrode layer orifice 126 can be coincident with a nanowell 150. An electrode layer orifice 126 is associated with a corresponding bore anterior opening 110 in nanowell chip 100. An electrode layer orifice 126 can be aligned with a substrate bore anterior opening 110, and the center point of an electrode layer orifice 126 can be concentric or about concentric with the center point of an associated and corresponding bore anterior opening 110 in nanowell chip 100 (with each center point determined for each member along radial direction 407 and with the center points for the members axially aligned along axial direction 405, for example). In alternative embodiments in which a nanowell chip includes electrode layer 120, (i) electrode layer 120 typically is not a reference electrode or working electrode when assembly 90 is utilized to analyze an analyte; and/or (ii) electrode layer 120 can serve as an electrode for monitoring fabrication of nanowell chip 100 as described herein.

Exemplary nanowell chip 100 includes insulator layer 130 (also referred to as an insulator film) that includes insulator layer anterior surface 132 and insulator layer posterior surface 134. An insulator layer can be a patterned insulator layer. A portion of insulator layer anterior surface 132 is disposed on, and optionally is bonded or adhered to, electrode layer anterior surface 122 of optional electrode layer 120 in nanowell chip 100. A separate portion of insulator layer anterior surface 132 is disposed on, and optionally is bonded or adhered to, substrate anterior surface 114 where there is no optional insulator layer 120 disposed on substrate 102. Insulator layer 130 can be patterned and can include a plurality of insulator layer orifice 136. An insulator layer orifice 136 can have any suitable geometry (as described for a nanowell herein) and can be circular for example. An insulator orifice 136 is associated with, and often is aligned with, a corresponding bore anterior opening 110 in nanowell chip 100. The center point of an insulator orifice 136 can be concentric or about concentric with the center point of the associated and corresponding bore anterior opening 110 in nanowell chip 100 (with each center point determined for each member along radial direction 407 and with the center points for the members axially aligned along axial direction 405, for example). An insulator orifice 136 also is associated with, and often is aligned with, a corresponding electrode layer orifice 126 in nanowell chip 100. The center point of an insulator orifice 136 can be concentric or about concentric with the center point of the associated and corresponding electrode layer orifice 126 in nanowell chip 100 (with each center point determined for each member along radial direction 407 and with the center points for the members axially aligned along axial direction 405, for example). An insulator layer orifice 136 can be coincident with a nanowell 150.

Exemplary nanowell chip 100 includes porous layer 140 (also referred to as a porous film) that includes porous layer posterior surface 142 and porous layer anterior surface 144. A portion of porous layer anterior surface 144 contacts, and optionally is bonded or adhered to, a portion of optional electrode layer posterior surface 124. A plurality of porous layer 140 can be included in nanowell chip 100 and each often is associated with, and can be aligned with, a corresponding substrate bore anterior opening 110. The center point of porous layer 140 can be concentric or about concentric with the center point of an associated and corresponding substrate bore anterior opening 110 in nanowell chip 100 (with each center point determined for each member along radial direction 407 and with the center points for the members axially aligned along axial direction 405, for example). A side of porous layer 140 can abut, or can be adjacent to and spaced a distance from, an edge of a corresponding substrate bore anterior opening 110, 110′. A porous layer 140 often is associated with, and can be aligned with, a corresponding insulator layer orifice 136 and/or optional electrode layer orifice 126. The center point of porous layer 140 can be concentric or about concentric with the center point of the associated and corresponding electrode layer orifice 126 and/or corresponding insulator layer orifice 136 in nanowell chip 100 (with each center point determined for each member along radial direction 407 and with the center points for the members axially aligned along axial direction 405, for example). A porous layer 140 often is associated with, and can be aligned with, a corresponding nanowell 150, and the center point of porous layer 140 can be concentric or about concentric with the center point of the associated and corresponding nanowell 150 (with each center point determined for each member along radial direction 407 and with the center points for the members axially aligned along axial direction 405, for example).

Exemplary nanowell chip 100 can include a single or plurality of nanowell 150. Each nanowell 150 typically includes nanowell anterior opening 152, nanowell posterior terminus 154 and nanowell sidewall 156. Nanowell anterior opening 152 and an anterior portion of nanowell sidewall 156 in nanowell chip 100 are defined by insulator orifice 136. A posterior portion of nanowell sidewall 156 is defined by electrode layer orifice 126, and nanowell posterior terminus 154 also is defined by electrode layer orifice 126 (defining the nanowell posterior terminus 154 perimeter) and a portion of porous layer anterior surface 144 not in contact with electrode layer posterior surface 124 (defining the nanowell posterior terminus 154 floor) in nanowell chip 100. A nanowell 150 in nanowell chip 100 generally is cylindrical, with a generally circular nanowell anterior opening 152, generally circular nanowell posterior terminus 154 and a nanowell sidewall 156 generally perpendicular to porous layer anterior surface 144 (and generally parallel to axial direction 405 for example). In nanowell chip 100 an anterior portion of nanowell 150 is defined by cylindrical insulator orifice 136 and a posterior portion of nanowell 150 is defined by cylindrical electrode layer orifice 126 having the same or substantially same diameter as concentric insulator orifice 136. Nanowell 150 can have a non-cylindrical geometry in alternative embodiments, defined by a non-cylindrical geometry of insulator orifice 136 and/or a non-cylindrical geometry of optional electrode layer orifice 126. A porous layer 140 often is associated with a corresponding nanowell 150. The center point of porous layer 140 can be concentric or about concentric with the center point of the associated and corresponding nanowell 150 (with each center point determined for each member along radial direction 407 and with the center points for the members axially aligned along axial direction 405, for example).

Exemplary partition member 200 includes partition member posterior surface 202, partition member anterior surface 204, and partition member exterior wall 208. Partition member 200 typically includes a plurality of partition member cavity 210. Each partition member cavity 210 is associated with a corresponding substrate bore posterior opening 106 in nanowell chip 100. The center point of a partition member cavity 210 at partition member anterior surface 204 can be concentric or about concentric with the center point of the associated and corresponding substrate bore posterior opening 106 in nanowell chip 100 (with each center point determined for each member along radial direction 407 and with the center points for the members axially aligned along axial direction 405, for example). A partition member cavity 210 includes a partition member interior wall 206. Each partition member cavity 210 in partition member 200 is cylindrical, with a circular opening at each of the partition member posterior surface 202 and partition member anterior surface 204, and with the partition member interior wall 206 generally perpendicular to the partition member posterior surface 202 (and generally perpendicular to substrate posterior surface 112 of nanowell chip 100 and generally perpendicular to electrode chip substrate posterior surface 306). A partition member cavity 210 can be non-cylindrical in alternative embodiments. Partition member anterior surface 204 contacts substrate posterior surface 112 of nanowell chip 100 in assembly 90. Partition member 200 often is connected to nanowell chip 100 by compression (by a clamp or cup exerting a compression force), or optionally can be bonded or adhered to nanowell chip 100 by an adhesive for example.

Exemplary electrode chip 300 includes substrate 302, separated electrode 320 (also referred to as working electrode) and intermediary layer 340. Substrate 302 includes substrate anterior surface 304 and substrate posterior surface 306. Electrode 320 includes electrode anterior surface 322 and electrode posterior surface 324.

Intermediary layer 340, which can be an electrode layer or metallic layer, includes intermediary layer anterior surface 342 and intermediary layer posterior surface 344. In alternative embodiments in which intermediary layer 340 is an electrode layer, (i) intermediary layer 340 typically is not a reference electrode or working electrode when assembly 90 is utilized to analyze an analyte; and/or (ii) intermediary layer 340 can serve as an electrode for monitoring fabrication of electrode chip 300. Intermediary layer posterior surface 344 contacts, and optionally is bonded or adhered to, substrate anterior surface 304 in electrode chip 300. A portion of intermediary layer anterior surface 342 contacts, and optionally is bonded or adhered to, electrode posterior surface 324 in electrode chip 300.

Intermediary layer 340 can be patterned in electrode chip 300 and can include a plurality of intermediary layer orifice 346. Electrode 320 can be patterned in electrode chip 300 and can include a plurality of electrode orifice 326. An electrode orifice 326 can be associated with a corresponding intermediary layer orifice 346. An electrode orifice 326 can include a center point that is concentric with the center point of a corresponding intermediary layer orifice 346 (with each center point determined for each member along radial direction 407 and with the center points for the members axially aligned along axial direction 405, for example). An electrode orifice 326 and corresponding intermediary layer orifice 346 can have the same geometry, and can have the same shape (circular or rectangular for example) and width(s), for example.

Electrode chip 300 connects to partition member 200, and an anterior portion of electrode chip 300 typically contacts a posterior surface of partition member 200. Partition member 200 often is connected to electrode chip 300 by compression (by a clamp or cup exerting a compression force, which can be the same device that exerts a compression force to nanowell chip 100), or optionally can be bonded or adhered to electrode chip 300 by an adhesive for example. A portion of a partition member posterior surface 202 can contact a portion of intermediary layer anterior surface 342 of electrode chip 300, and/or a portion of a partition member posterior surface 202 can contact a portion of substrate anterior surface 304 of electrode chip 300. In electrode chip 300, an electrode orifice 326 and corresponding intermediary layer orifice 346 together can form a depression 308 having sides defined by the orifices 326 and 346 and a floor defined by an exposed portion of substrate anterior surface 304. Electrode chip 300 can include a plurality of depression 308. A depression 308 can receive a corresponding portion 212 of partition member 200, where substrate anterior surface 304 within depression 308 contacts posterior surface 202 of corresponding portion 212, and portion of a side of portion 212 optionally contacts a side of orifice 326 and/or 346 or is spaced a distance from a side of 326 and/or 346.

Nanowell chip 100, partition member 200 and electrode chip 300 in assembly 90 are oriented such that an electrode 320 is in electroconductive association with a corresponding nanowell 150. Nanowell chip 100, electrode chip 300 and partition member 200 in assembly 90 are oriented such that one electrode 320 is in electroconductive association with a corresponding nanowell 150, and that the same nanowell is not in electroconductive association with another working electrode of the electrode chip 300. For example, nanowell 150a is in electroconductive association with electrode 320a via partition member cavity 210a, and nanowell 150b is in electroconductive association with electrode 320b via partition member cavity 210b. Nanowell 150a is not in electroconductive association with electrode 320b, nanowell 150b is not in electroconductive association with electrode 320a, and nanowell 150a is not in electroconductive association with nanowell 150b, due to electroconductive separation of partition member cavity 210a and 210b in assembly 90. In assembly 90, electrode 320a can be in electroconductive association with a single nanowell 150a or a plurality of nanowell 150a, and electrode 320b can be in electroconductive association with a single nanowell 150b or a plurality of nanowell 150b.

The nanowell chip illustrated in FIG. 1A and FIG. 1B includes a substrate that can contain one or more of glass, sapphire, ceramic, oxide-coated silicon, polycarbonate and polyimide, for example. A substrate sometimes is or includes an isotropic or anisotropic anodic aluminum oxide wafer or anodic aluminum oxide grown on the surface of another material. A substrate sometimes is about 20 to about 2000 micrometers thick (in axial direction 405 for example), and can comprise a maximum thickness of about 20 micrometers to about 100 micrometers, or about 75 micrometers to about 500 micrometers, or about 250 micrometers to about 5000 micrometers.

A substrate can be a patterned substrate and can include one or more patterned bores. In alternative embodiments, a bore is disposed on a posterior surface of a substrate and extends to an anterior surface of the substrate. Bores, which may be holes, channels, domes, or other geometries described, typically penetrate entirely through the substrate under each nanowell while only exposing a minimal area of the underside of each electrode at the top surface in order to minimize the capacitance of the system. A bore interior wall of can be curved or angled. A bore can be aligned with a nanowell of a chip and a bore can be concentric with a corresponding nanowell. A virtual axial center point of a nanowell can be aligned with and coincident with a virtual axial center point of a substrate bore in alternative embodiments. A bore can have an anterior opening at the substrate anterior surface, can have a posterior opening at the substrate posterior surface, and can have a depth (in axial direction 405 for example) equal to the substrate thickness. A bore can have a width (in radial direction 407 for example) of about 1 micrometer to about 500 micrometers, or about 1 micrometer to about 50 micrometers, or about 20 to about 250 micrometers, or about 100 micrometers to about 5000 micrometers at the substrate anterior surface, and/or a width of about 1 micrometer to about 2000 micrometers, or about 1 micrometer to about 50 micrometers, or about 25 micrometers to about 500 micrometers, or about 250 micrometers to about 20000 micrometers at the substrate posterior surface.

In alternative embodiments, a nanowell chip comprises an insulator film, which also is referred to as an insulator layer and which can contain one or more of an epoxy (for example, bisphenol A Novolac epoxy (for example, SU8)), polyimide, parylene, photoresist, polystyrene, fluoropolymer, silicon dioxide, silicon nitride, for example. An insulator layer surface area can be less than the surface area of the substrate. In alternative embodiments, an insulator layer surface area is equal to the surface area or greater than of the substrate. An insulator layer can have a maximum thickness (in axial direction 405 for example) of about 50 nanometers to about 5000 nanometers, and a minimum thickness (in axial direction 405 for example) of about 50 nanometers to about 500 nanometers, or about 250 nanometers to about 1000 nanometers, or about 750 nanometers to about 5000 nanometers, or about 5000 nanometers to about 100,000 nanometers. An insulator layer corresponding to a particular nanowell can have a width (in radial direction 407 for example) of about 50 nanometers to about 5000 nanometers, or about 50 nanometers to about 500 nanometers, or about 250 nanometers to about 1000 nanometers, or about 750 nanometers to about 5000 nanometers, or about 5000 nanometers to about 100,000 nanometers. An insulator material may include a combination of hydrophobic and hydrophilic regions. An insulator film can include pillars and/or cutouts or different structures. An insulator layer can be a patterned insulator layer. An insulator layer can include a single or a plurality of orifice or through hole defining each nanowell developed down to the porous film. An insulator layer can include a cutout, orifice or hole corresponding to each nanowell in a nanowell chip. An opening of an insulator layer cutout, orifice or hole at an insulator layer surface can be any suitable shape, such as circular, oval, rectangular, square or triangular for example. An insulator cutout, orifice or hole can be aligned with and/or coincident with a nanowell, and can delimit a nanowell or delimit a portion of a nanowell, such as an anterior portion of a nanowell for example.

In alternative embodiments, a nanowell chip comprises an optional electrode material or materials joined to an anterior surface of the substrate, which is referred to as an electrode layer. An electrode layer can be a metallic layer. An electrode layer disposed on a substrate can include one or more of gold, chrome, indium tin oxide (ITO), platinum, and titanium nitride, for example. An electrode layer deposited on a substrate of a nanowell chip sometimes is about 5 nanometers (nm) to about 1000 nm thick (in axial direction 405 for example), which can be a maximum thickness of about 5 nanometers to about 100 nanometers, or about 20 nanometers to about 200 nanometers, or about 100 nanometers to about 1000 nanometers, or about 1000 nanometers to about 10000 nanometers and can be patterned. An electrode layer can include a through hole defining each nanowell developed down to the porous film. An electrode layer can include pillars and/or cutouts or different structures. An electrode layer can be a patterned electrode layer and can include a cutout or hole corresponding to each nanowell in a nanowell chip. An electrode layer cutout or hole at an electrode layer surface can be any suitable shape, such as circular, oval, rectangular, square or triangular for example. An electrode cutout or hole can be aligned with and/or coincident with a nanowell, and can delimit a nanowell or delimit a portion of a nanowell, such as a posterior portion of a nanowell for example. An electrode layer, when present in a nanowell chip, often does not serve as a working electrode and often does not serve as a reference electrode in a nanowell chip assembly used to analyze an analyte, and can serve as an electrode member for monitoring substrate bore break through in a chip manufacturing process, as described.

An optional electrode layer surface area can be less than the surface area of the substrate, and can be less than the surface area of the insulator layer. An insulator layer can be disposed on an anterior surface of the electrode material of a nanowell chip that includes an optional electrode layer. An anterior surface of an optional electrode layer can contact a posterior surface of the insulator layer and a posterior surface of the optional electrode layer can contact an anterior surface of the nanowell chip substrate. For a nanowell chip that does not include an optional electrode layer, the posterior surface of an insulator film can be disposed entirely on a substrate anterior surface. A nanowell chip can include a discontinuous electrode layer disposed on a nanowell chip substrate, a portion of an insulator layer can be disposed on the electrode layer and a portion of the insulator film can be disposed on a portion of the substrate at which the electrode layer is not disposed. In the latter embodiment, an insulator layer thickness above the substrate typically is greater than the insulator layer thickness above the electrode layer, and the difference in the insulator layer thickness above the substrate and above the electrode typically is equal to or about equal to the electrode layer thickness.

In alternative embodiments, a porous film, also referred to as a porous layer, can be disposed on a posterior surface of an optional electrode material of a nanowell chip. For a nanowell chip not including an optional electrode layer, a porous layer can be disposed on a posterior surface of an insulator film. A porous film often is bounded by the substrate bore anterior opening, and often includes or is a selectively permeable layer or film often containing an array of vertically aligned pores.

In alternative embodiments, a selectively permeable porous film transmits supporting electrolyte through the pores, and often precludes transmission or blocks transmission of an analyte of interest through the pores. A supporting electrolyte typically is an electrolyte that conducts electric current through a nanopore reader in a system, sometimes is an ion that migrates towards and discharges at a negative or positive electrode in a system, and sometimes is or part of an acid, base or salt. An analyte of interest, which also is referred to herein as an analyte, sometimes is a biopolymer monomer unit (for example, a nucleoside, nucleotide, amino acid, modified monomer unit, modified nucleoside, modified nucleotide, modified amino acid), biopolymer, modified biopolymer, nucleic acid, modified nucleic acid, peptide, modified peptide, polypeptide, modified polypeptide, protein, modified protein, virus, modified virus, nanoparticle, modified nanoparticle, liposome, modified liposome, vesicle, modified vesicle and the like.

A porous film can include one or more of mesoporous silica, mesoporous titania, or anodic aluminum oxide, a molecular thin film (for example, graphene, graphene oxide), a polymer (for example, polyvinyl chloride, polyetheretherketone, block copolymer), a transition metal carbide or nitride, boron nitride, carbon nanotubes, molybdenum disulfide, chemically modified glass frit, sol-gel, chemically modified sol-gel, metal organic framework, and a solid-state nanopore (for example, solid state nanopore in silicon nitride via transmission electron microscopy (TEM) drilling, focused ion beam, or dielectric breakdown). A porous film may be functionalized by silane chemistry or coated via atomic layer deposition to achieve desired permeability.

A porous layer can have a thickness (in axial direction 405 for example) of about 10 nanometers to about 10000 nanometers, or about 10 nanometers to about 150 nanometers, or about 50 nanometers to about 1000 nanometers, or about 750 nanometers to about 10000 nanometers. A porous layer corresponding to a particular nanowell can have a width (in radial direction 407 for example) of about 50 nanometers to about 10000 nanometers, or about 50 nanometers to about 500 nanometers, or about 250 nanometers to about 1500 nanometers, or about 1000 nanometers to about 10000 nanometers, or about 10000 nanometers to about 100000 nanometers. The surface area covered by the porous film may be about 0.2 to about 200000 square micrometers, about 1 to 10000 square micrometers, about 1000 to about 8000 square micrometers, or about 50, 100, 200, 500, 1258, 7850, 18000, 31400, or 70650 square micrometers. A porous layer can have the same or about the same width as a substrate bore width at the substrate anterior surface, or a porous layer can have a width less than the substrate bore width at the substrate anterior surface.

A nanowell chip can be provided in an assembly containing an electrode chip and an optional partition member. In alternative embodiments, an electrode chip typically comprises an insulating substrate. An insulating substrate can include one or more of glass, sapphire, ceramic, oxide-coated silicon, polycarbonate, polyimide, printed circuit board, for example. An insulating substrate sometimes is about 20 to about 2000 microns thick. An electrode material or materials often are joined to an anterior surface of the substrate of an electrode chip, and can contain one or more of gold, chrome, indium tin oxide (ITO), platinum, titanium, silver, and titanium nitride, for example. An electrode material sometimes is about 5 (nanometers) nm to about 1000 nm thick (in axial direction 405 for example), or an electrode material can comprise a thickness of about 5 nm to about 100 nm, or about 50 nm to about 500 nm, or about 250 nm to about 1000 nm, or about 1000 nm to 10000 nm, and can be patterned in an electrode chip. An electrode material of an electrode chip can comprise a width of about 10 micrometers (μm) or greater, or about 100 μm or greater, or about 1,000 μm or greater, or about 5 micrometers to about 150 micrometers, or about 100 micrometers to about 500 micrometers, or about 250 micrometers to about 1000 micrometers (in radial direction 407 for example). The electrodes of an electrode chip can include leads that run to the edge of the chip with contact pads. The substrate can include a conductor leading to the posterior surface of the chip, which can serve as through vias for interfacing with the control electronics. An electrode chip can include a working electrode, or a reference electrode, or a working electrode and a reference electrode. A working electrode and a reference electrode each independently can be a disk and can have a circular surface.

In alternative embodiments, a partition member can be an insulating partition member. A partition member can include one or more of polydimethylsiloxane (PDMS), silicone, acrylic, polycarbonate, polytetrafluoroethylene, and a fluoropolymer, with or without adhesives. A partition member can be aligned and sandwiched between a nanowell chip and electrode chip such that a fluidic connection between individual nanowells and their underlying micro-/macroelectrode are isolated from adjacent nanowells (for example, the electrodes in the electrode chip are separated from the nanowells, spaced a distance from the nanowells, and are not disposed in the nanowells of a nanowell chip). A partition member typically includes a plurality of voids. A partition member sometimes is a gasket containing a plurality of orifices, with each orifice often traversing the entire thickness of gasket and typically configured to associate with a nanowell of a nanowell chip and a corresponding electrode (for example, a working electrode) of an electrode chip. A partition member sometimes contains a plurality of fluidic channels, with each fluidic channel configured to associate with a nanowell of a nanowell chip and a corresponding electrode (for example, a working electrode) of an electrode chip. In certain implementations, a partition member can include two partition member components, with a first component joined to a posterior surface of a nanowell chip and a second component joined to an anterior surface of an electrode chip. The first component and the second component can be joined in a system or assembly. A partition member can be about 10 micrometers to about 1000 micrometers thick (in axial direction 405 for example), or comprise a thickness of about 10 micrometers to about 100 micrometers, or about 50 micrometers to about 500 micrometers, or about 250 micrometers to about 10000 micrometers.

Layers in each of a nanowell chip, electrode chip and/or partition member can be bonded to one another. Bonding can be chemical bonding and/or adhesive bonding. A nanowell chip, electrode chip and/or partition member in an assembly can be reversibly or irreversibly connected to one another. Members of an assembly containing a nanowell chip, electrode chip and/or partition member can bonded, such as by an adhesive for example. Members of an assembly containing a nanowell chip, electrode chip and/or partition member can be connected by compression. Compression can be applied by a clamp or cup in association with two or more of the assembly members (the nanowell chip and the electrode chip for example) or another suitable device. A non-limiting example of a clamp is illustrated in FIG. 1C. In FIG. 1C, clamp 410 includes first clamp member 412 that includes clamping surface 414 that contacts a posterior surface of electrode chip 300. Claim 410 also includes hinge member 416 between first clamp member 412 and second clamp member 418. Second clamp member 418 includes clamping surface 420 that contacts and deforms deformable clamp intermediate 430 against an anterior surface of nanowell chip 100 (an anterior surface of the insulator layer of nanowell chip 100 for example). Clamp intermediate 430 can be a gasket and/or o-ring and can be constructed from any suitable deformable material, which sometimes is a resilient material. Clamp 410 can be constructed from any suitable rigid material, such as a rigid polymer and/or a metal for example. Hinge member 416 can include a spring or can be a spring that exerts a downward force on second clamp member clamping surface 420 towards first clamp member clamping surface 414, thereby joining components of assembly 90 by a compression force.

FIG. 2A to FIG. 2F show non-limiting examples of the nanowell structure with the substrate cut outs and openings having curved or straight walls, or having the substrate replaced with a porous substrate like anodic aluminum oxide for example. FIG. 2A and FIG. 2B illustrate a nanowell (550, 650) in a nanowell chip (500, 600) that includes an insulator layer (530, 630) disposed on an electrode layer (520, 620) anterior surface, the electrode layer (520, 620) posterior surface disposed on a substrate (502, 602), and a porous film (540, 640) anterior surface disposed on the electrode layer (520, 620) posterior surface. A bore extending from the substrate (502, 602) posterior surface to substrate anterior surface can be a dome with a curved interior wall as illustrated in FIG. 2A or can include an angled interior surface as illustrated in FIG. 2B. The sidewall of the nanowell (550, 640) illustrated in FIG. 2A and FIG. 2B is defined by a hole in the insulator layer (530, 630) and a concentric hole having the same diameter in the electrode layer (520, 620), and the floor of the nanowell coincides with the anterior surface of the porous layer (540, 640) disposed below the hole of the electrode layer (520, 620). FIG. 2C illustrates a nanowell (750) in a nanowell chip (700) that includes an insulator layer (730) disposed on an electrode layer (720) anterior surface, the electrode layer (720) posterior surface disposed on a substrate (702), an intermediary layer that includes an active layer (762) and support layer (764), and a porous substrate layer (760) anterior surface disposed on a posterior surface of the intermediary layer (posterior surface of layer 764). The sidewall of nanowell (750) illustrated in FIG. 2C is defined by a hole in the insulator layer (730) and a concentric hole having the same diameter in the electrode layer (720), and the floor of the nanowell coincides with the anterior surface of the active layer (762) disposed below the hole of the electrode layer (720).

FIG. 2D and FIG. 2E illustrate a nanowell (850, 950) in a nanowell chip (800, 900) that includes an insulator layer (830, 930) disposed on a substrate (802, 902) posterior surface, without an intermediary electrode layer, and a porous film (840, 940) anterior surface disposed on the insulator layer (830, 930) posterior surface. A bore extending from the substrate (802, 902) posterior surface to substrate anterior surface can be a dome with a curved interior wall as illustrated in FIG. 2D or can include an angled interior surface as illustrated in FIG. 2E. The sidewall of the nanowell (850, 940) illustrated in FIG. 2D and FIG. 2E is defined by a hole in the insulator layer (830, 930) and the floor of the nanowell coincides with the anterior surface of the porous layer (840, 940) disposed below the hole of the insulator layer (830, 930). FIG. 2F illustrates a nanowell (1050) in a nanowell chip (1000) that includes an insulator layer (1030) disposed on a substrate (1002) anterior surface, an intermediary layer that includes a active layer (1062) and support layer (1064), and a porous substrate layer (1060) anterior surface disposed on a posterior surface of the intermediary layer (posterior surface of layer 1064). The sidewall of nanowell (1050) illustrated in FIG. 2E is defined by a hole in the insulator layer (1030, and the floor of the nanowell (1050) coincides with the anterior surface of the active layer (1062) disposed below the hole of the insulator layer (1030).

For embodiments in which a nanowell chip includes a porous substrate (as illustrated in FIG. 2C and FIG. 2F for example), a porous substrate layer (layer 760 or 1060 for example) can comprise a thickness of about 20 micrometers to about 2000 micrometers, or about 20 micrometers to about 100 micrometers, or about 75 micrometers to about 500 micrometers, or about 250 micrometers to about 2000 micrometers. A porous substrate can include one or more of anodic aluminum oxide, ion track etched polymers (e.g., polycarbonate, polyethylene terephthalate, or polyimide. An active layer (layer 762 or 1062 for example) can comprise a thickness of about 0.5 micrometer to 20 micrometers (in axial direction 405 for example), or about 0.5 micrometer to about 2 micrometers, or about 1 micrometer to about 10 micrometers, or about 5 micrometers to about 20 micrometers. An active layer can include one or more of anodic aluminum oxide, block copolymer films, mesoporous silica, metal organic frameworks, polyvinylidene fluoride, polyacrylonitrile, polystyrene and polysulfone. A support layer (layer 764 or 1064 for example) can comprise a thickness of about 1 micrometer to about 500 micrometers (in axial direction 405 for example), or about 1 micrometer to about 20 micrometers, or about 10 micrometers to about 200 micrometers, or about 150 micrometers to about 500 micrometers. A support layer can include one or more of anodic aluminum oxide, block copolymer films, mesoporous silica, metal organic frameworks, polyvinylidene fluoride, polyacrylonitrile, polystyrene and polysulfone.

FIG. 3A to FIG. 3D illustrate exemplary assemblies containing a nanowell chip, working electrode and reference electrode. FIG. 3A, FIG. 3B and FIG. 3C illustrate exemplary assemblies containing a partition member and electrode chip.

Exemplary nanowell chip assembly 1100 illustrated in FIG. 3A includes nanowell chip 1101, partition member 1170, electrode chip 1190, working electrode 1180 and reference electrode 1185. Nanowell chip 1101 includes substrate 1102, electrode layer 1120, insulator layer 1130, porous layer 1140 and nanowell 1150. Electrode chip 1190 includes electrode chip substrate 1192 and electrode chip intermediary layer 1195. Partition member 1170 and electrode chip 1190 are disposed in a similar orientation as described for assembly 90 illustrated in FIG. 1A and FIG. 1B. Working electrode 1180 is disposed on a portion of intermediary layer 1195 of electrode chip 1190 and reference electrode 1185 is disposed on a separate portion of intermediary layer 1195 of electrode chip 1190. The separate portions of intermediary layer 1195 on which reference electrode 1185 and working electrode 1180 are disposed are discontinuous and separated by partition member 1170. Working electrode 1180 is disposed within a partition member cavity and is in electroconductive association with corresponding nanowell 1150. Reference electrode 1185 and working electrode 1180 are connected by electrical circuit 1197. There is no substrate 1102 portion disposed in an anterior position relative to reference electrode 1185 in assembly 1100.

Exemplary nanowell chip assembly 1200 illustrated in FIG. 3B includes nanowell chip 1201, partition member 1270, electrode chip 1290, working electrode 1280 and reference electrode 1285. Nanowell chip 1201 includes substrate 1202, electrode layer 1220, insulator layer 1230, porous layer 1240 and nanowell 1250. Electrode chip 1290 includes electrode chip substrate 1292 and electrode chip intermediary layer 1295. Partition member 1270 and electrode chip 1290 are disposed in a similar orientation as described for assembly 90 illustrated in FIG. 1A and FIG. 1B. Working electrode 1280 is disposed on a portion of intermediary layer 1295 of electrode chip 1290 and reference electrode 1285 is disposed on a separate portion of intermediary layer 1295 of electrode chip 1290. The separate portions of intermediary layer 1295 on which reference electrode 1285 and working electrode 1280 are disposed are discontinuous and separated by partition member 1270. Working electrode 1280 is disposed within a partition member cavity and is in electroconductive association with corresponding nanowell 1250. Reference electrode 1285 and working electrode 1280 are connected by electrical circuit 1297. A portion of substrate 1202 is disposed in an anterior position relative to reference electrode 1285 in assembly 1200.

Exemplary nanowell chip assembly 1300 illustrated in FIG. 3C includes nanowell chip 1301, partition member 1370, electrode chip 1390, working electrode 1380 and reference electrode 1385. Nanowell chip 1301 includes substrate 1302, electrode layer 1320, insulator layer 1330, porous layer 1340 and nanowell 1350. Electrode chip 1390 includes electrode chip substrate 1392 and electrode chip intermediary layer 1395. Partition member 1370 and electrode chip 1390 are disposed in a similar orientation as described for assembly 90 illustrated in FIG. 1A and FIG. 1B. Working electrode 1380 is disposed on a portion of intermediary layer 1395 of electrode chip 1390. Reference electrode 1385 is not disposed on a portion of intermediary layer 1395 of electrode chip 1390 and instead is disposed separately in an anterior position, and cis position, relative to the insulator layer 1330 anterior surface. Working electrode 1380 is disposed within a partition member cavity and is in electroconductive association with corresponding nanowell 1350. Reference electrode 1385 and working electrode 1380 are connected by electrical circuit 1397.

FIG. 3D illustrates an exemplary assembly not containing a partition member and electrode chip. Exemplary nanowell chip assembly 1400 illustrated in FIG. 3D includes nanowell chip 1401, working electrode 1480 and reference electrode 1485, and not partition member and no electrode chip. Nanowell chip 1401 includes substrate 1402, electrode layer 1420, insulator layer 1430, porous layer 1440 and nanowell 1450. Working electrode 1480 is disposed separately in a posterior position, and cis position, relative to the insulator layer 1430 anterior surface, and in a posterior position relative to the substrate 1402 posterior surface. Working electrode 1480 is in electroconductive association with corresponding nanowell 1450. Reference electrode 1485 is disposed separately in an anterior position, and cis position, relative to the insulator layer 1430 anterior surface. Reference electrode 1485 and working electrode 1480 are connected by electrical circuit 1497.

As illustrated in FIG. 3A to FIG. 3D, a reference electrode can be disposed in a cis position relative to a nanowell anterior opening as depicted in FIG. 3C and FIG. 3D, can be disposed in bulk solution, separate from the nanowell chip, partition membrane, and electrode chip, or can be disposed on the electrode chip (as depicted in FIG. 3A and FIG. 3B), disposed in a posterior position relative to the nanowell chip.

In alternative embodiments, one or more reference electrodes, which can include one or more of silver (for example, silver/silver chloride, silver/silver sulfate), calomel, iridium oxide, copper/copper sulfate, gold and platinum, can be disposed in a cis position relative to a nanowell anterior opening as depicted in FIG. 3C and FIG. 3D, can be disposed in bulk solution, separate from the nanowell chip, partition membrane, and electrode chip, or can be disposed on the electrode chip (as depicted in FIG. 3A and FIG. 3B), disposed in a posterior position relative to the nanowell chip. Redox couples, including ferri/ferrocyanide and/or ruthenium hexamine, for example, dissolved in solution may also be used in conjunction with bare metal electrodes, such as gold or platinum, to provide a reference or working electrode system. In alternative embodiments, the reference electrode or electrodes can be the working electrode or electrodes, and the working electrode or electrodes can be the reference electrode or electrodes, as would be determined by how the electrode chip and electrodes are connected to the measurement apparatus, system or electronics.

A nanowell chip can contain a phospholipid bilayer (PLB) or other membrane in association with a nanowell. A nanowell chip illustrated herein (in FIG. 1A, FIG. 1B, FIG. 2A to FIG. 2F, FIG. 3A to FIG. 3D or FIG. 6A to FIG. 6G, can include a PLB or other membrane in association with one nanowell or a plurality of nanowells, and optionally can contain a nanopore reader. A nanowell of a nanowell chip also can contain one or more proteins that process a polymer, such as an enzyme for example (a nuclease, exonuclease, polymerase or other enzyme for example).

An exemplary PLB or membrane over the anterior opening of the well within the insulator layer of the nanowell chip is illustrated in FIG. 5A and FIG. 5B. An exemplary nanowell chip assembly 1500 is illustrated in FIG. 5A and an expanded view of the portion shown within the broken-line box in FIG. 5A is illustrated in FIG. 5B. Exemplary nanowell chip assembly 1500 includes nanowell chip 1501, partition member 1570, electrode chip 1590, and working electrode 1580. Nanowell chip 1501 includes substrate 1502, electrode layer 1520, insulator layer 1530, porous layer 1540 and nanowell 1550. Electrode chip 1590 includes electrode chip substrate 1592, electrode chip intermediary layer 1595 and working electrode 1580 disposed on intermediary layer 1595. With partition member 1570 disposed in an anterior position and in contact with electrode chip 1590, working electrode 1580 is disposed within a cavity of partition member 1570 and is in electroconductive association with corresponding nanowell 1550. As illustrated in FIG. 5B, lipid bilayer membrane 1566 is associated with nanowell 1550, and includes first lipid layer 1567 and second lipid layer 1568. A flanking portion of first lipid layer 1567 is disposed on a portion of insulator layer 1530 anterior surface surrounding the anterior opening of nanowell 1550. A flanking portion of second lipid layer 1568 is disposed on a portion of the interior wall of nanowell 1550, and a separate central portion of second lipid layer 1568 is in association with first lipid layer 1567 forming bilayer membrane 1566.

In alternative embodiments, a phospholipid bilayer (PLB) or other membrane is formed over and/or within each nanowell of the chip, using known methods to paint or cast thin films of membrane-forming materials over the well. A PLB or other membrane often is formed over and/or within each nanowell after the nanowell chip is fabricated and prior to using the nanowell chip to analyze an analyte. A portion of a PLB or other membrane can contact a portion of an insulator layer anterior surface surrounding a nanowell opening. A portion of a PLB or other membrane can contact an interior sidewall surface of a nanowell. A PLB or other membrane over a nanowell sometimes contains a nanopore reader, which may be incorporated into the PLB or other membrane using known processes after the nanowell chip is fabricated and prior to using the nanowell chip to analyze an analyte. A PLB or other membrane over a nanowell sometimes contains no nanopore reader. While a PLB is referred to here over each well, any suitable membrane seal capable of retaining an individual nanopore reader can be utilized, including but not limited to a seal comprising phospholipids (e.g. DPhPC, POPC, DOPC, DMPC, DoPhPC) or mixtures thereof, surfactants, di-block copolymers (e.g. polybutadiene-polyethylene oxide), tri-block copolymers (e.g. poly-2-methyl-2-oxazoline-polydimethylsiloxane-poly-2-methyl-2-oxazoline), mycolic acid, mixtures thereof, or polymerizable versions thereof.

In alternative embodiments, a nanopore reader can be disposed in nanowell of a nanowell chip. A nanopore reader can be disposed in a PLB or other membrane of a nanowell chip. A reader can be a protein containing a pore or channel. Non-limiting examples of biological nanopores, ion channels, or transmembrane proteins that can be utilized as readers include alpha-hemolysin (alpha-HL), aerolysin, Mycobacterium smegmatis porin A (MspA), Escherichia coli CsgG, Cytolysin A (ClyA), and outer membrane protein F (OmpF), NetB protein toxin, modified or mutant forms of secretin, and Fragaceatoxin C (FraC). Non-limiting examples of synthetic engineered biological nanopores include DNA nanopores (also referred to as “DNA-based nanopores” or “DNA origami nanopores”) and engineered peptide nanopores. Chemical crosslinking agents that covalently link individual subunits of a reader or that tune the performance of one or more readers also can be utilized.

Nanopore readers can be mutated or modified to contain additional charged residues that serve to alter the charge distribution on and within the nanopore reader to control the magnitude and direction of electroosmotic flow. For example alpha-HL can be modified with additional positive charged residues (K and R) throughout its beta-barrels (for example, T115K, T117R, G119K, and N121R) at positions that have been previously altered in order to enhance molecular transport through the pore. Similar modifications can be made in MspA: N79R, N86R, N108K or N121K in MspA. The cationic surface on the interior of the protein nanopore reader generates an excess of anionic counter ions within the pore, facilitating net flow into the interior of the pore under a positive applied voltage bias (trans to cis).

### Fabrication

A non-limiting example and generalized series of process steps for fabricating nanowell chips is illustrated in FIG. 4. The sequence in which these steps can in some cases be rearranged and still achieve the desired structure. A protective layer (for example, a photoresist, gold, silicon nitride, or combinations of these depending upon the substrate material and etchant used) can be patterned onto the bottom surface of the substrate leaving openings where material is to be removed. The same or other protective materials may be used for the top surface, such as adhesive tape with polymer backing or wax coating. Substrate cut outs/openings then can be developed through the substrate by chemical etching (for example, utilizing hydrofluoric acid, potassium hydroxide, and/or phosphoric acid) or deep reactive ion etching, and stopped just before breakthrough based upon elapsed time and a known etch rate. Protective layers then can be removed. Next, gold or other preferred metals can be deposited (for example, by sputtering, chemical vapor deposition, electron beam evaporation) on the top surface of the substrate and then patterned by a standard lithographic process (for example, by spin coat photoresist, develop photoresist, and/or etch metals or liftoff resist). Next, the insulator film can be grown, deposited, spin coated, 3D printed, or applied to the top surface and patterned by standard photo- or electron beam lithographic processes and/or plasma/reactive ion etching. Next, the remainder of substrate can be etched to complete the through holes. Only a small area of the top surface metallic layer typically is exposed, and etching may be stopped by timing it with a known etch rate or by electrically monitoring the top surface metallic layer for when breakthrough is detected (the latter is illustrated in FIG. 4).

In alternative embodiments, a porous film with selective permeability then can be grown on the underside of the top surface electrode layer. The porous film can be grown by a template assisted method where mixtures of a structure directing material (for example, a surfactant or block copolymer) and a guest material (for example, silica or titania sol gel) can be deposited together followed by removal of one or the other. Growth of the porous film can be achieved by electrochemical methods, chemical vapor deposition, and/or self-assembly. Non-limiting examples of electrochemical methods include aluminum anodization followed by etching with phosphoric acid to make anodic aluminum oxide, water reduction to change pH locally and drive condensation of sol gel based films (for example, mesoporous silica or titania), co-deposition of metals (for example, nickel, gold, silver) or polymers (for example, polyaniline polymer (PANI polymer), poly(3,4-ethylenedioxythiophene) polymer (PEDOT polymer), polypyrrole polymer (PPy polymer)) along with porogens that are subsequently removed. Non-limiting examples of chemical vapor deposition methods include co-deposition of monomers (for example, methacrylates) and initiators (for example, peroxides) with porogens (for example, siloxanes, naphthalene). Non-limiting examples of self assembly methods include random dense packing of nanoparticles, evaporation induced film formation via dip coating, spray coating, spin coating, or printing of precursors directly onto a surface. Pores may also be formed by TEM drilling, dielectric breakdown, or track etching. Metal organic frameworks and other crystalline polymer, inorganic-organic hybrid materials that are linked to metal ions by coordination bonds crosslinked by organic ligands also may be utilized.

The top surface metallic layer then can be etched from the bottom of the nanowell by introducing etchant (for example, iodine/iodide or cyanide/oxidizer) on the top surface. Extraction or etching of the template or porogen then can be performed to complete the nanowell chip fabrication.

The anterior electrode or metallic layer on the nanowell chip, when it is utilized, can be used (i) to electrically or electrochemically monitor the formation and enlargement of the bore through the substrate during its formation, and/or (ii) to electrochemically deposit the porous layer onto the posterior of the electrode inside of the bore, and/or (iii) as an attachment point inside of the confined nanowell volume, for instance in cases in which a Au electrode on nanowell chip is utilized, thiol based bioconjugation strategies can be used to directly attach individual molecules, layers, enzymes, and or groups of molecules or enzymes to the exposed Au walls of the nanowell, that have been etched through to expose the porous layer at the bottom of the nanowell.

Bores in a substrate of a nanowell chip can be of any suitable shape and formed by a suitable process. Dome-shaped bores (as illustrated in FIG. 1A, FIG. 1B, FIG. 2A and FIG. 2D for example) can be formed through or in a nanowell chip substrate by isotropic processes such as an acid etching process for example (etching of silicon dioxide by hydrofluoric acid, for example). Frustrum-shaped bores (as illustrated in FIG. 2B and FIG. 2E for example) can be fabricated by anisotropic processes such as a base etching process (KOH etching of silicon for example) or a laser process followed by acid or base etching (induced deep etching of a glass for example), for example.

Nanowell chips in some embodiments may be prepared without an intermediary electrode layer and can have an insulator layer directly on the substrate anterior substrate surface (for example, an insulator layer can be disposed entirely on an anterior substrate surface with no intermediary electrode layer disposed on all or part of the substrate anterior surface, as illustrated in FIG. 2D and FIG. 2E for example). In such embodiments, the porous layer can be grown by vapor deposition methods or deposited by dip coating prior to etching the nanowell instead of by electrochemical methods.

Nanowell chips can be fabricated with a porous substrate (as illustrated in FIG. 2C and FIG. 2F for example) instead of a non-porous substrate (as illustrated in FIG. 1A, FIG. 1B, FIG. 2A, FIG. 2B, FIG. 2D and FIG. 2E, for example). Porous substrates can include vertical channels such as channels produced in anodic aluminum oxide and these channels need not be the same size throughout the porous substrate. Voltage may be varied throughout an anodization process to produce larger pore sizes in the support layer while applying an appropriate voltage to achieve pore sizes appropriate for the nanowell confinement while growing the active layer.

In alternative embodiments, porous substrates, support layers, and active layers can be different materials. In alternative embodiments, an anodic aluminum oxide porous substrate may have block copolymer deposited on its surface that self assembles to form a support layer followed by dip coating with a sol gel for growth of a mesoporous silica active layer. In alternative embodiments, an electrode layer or an insulator layer may be deposited onto the active layer by sputtering, vapor deposition, or by spin coating.

In alternative embodiments, a nanowell chip can be manufactured by a process that includes: (a) providing a substrate comprising a substrate bore and an electrode layer joined to a substrate anterior surface, the substrate bore comprising a posterior bore opening disposed at a substrate posterior surface and an anterior terminus disposed within the substrate; (b) optionally patterning the electrode layer whereby an electrode layer portion is disposed in an anterior position over a substrate bore; (c) depositing an insulator layer on an anterior surface of the electrode layer; (d) introducing a hole within the insulator layer in an anterior position over a substrate bore, wherein the hole comprises an anterior hole opening disposed at an insulator layer anterior surface and a hole posterior terminus disposed at the electrode layer; (e) deepening the substrate bore, whereby the substrate bore comprises a bore anterior opening disposed at an electrode layer posterior surface; (f) depositing a porous layer on the electrode layer posterior surface at the bore anterior opening; and (g) etching the electrode layer disposed at the hole posterior terminus, thereby forming a nanowell comprising an anterior nanowell opening disposed at an insulator layer anterior surface and a nanowell posterior terminus disposed at a porous layer anterior surface. Non-limiting examples of nanowell chips that can be manufactured by such a process include those illustrated in FIG. 1A, FIG. 1B, FIG. 2A and FIG. 2B.

In alternative embodiments, a nanowell chip can be manufactured by a process that includes: (a) providing a substrate comprising a substrate bore, the substrate bore comprising a posterior bore opening disposed at a substrate posterior surface and an anterior terminus disposed within the substrate; (c) depositing an insulator layer on an anterior surface of the substrate; (d) deepening the substrate bore, whereby the substrate bore comprises a bore anterior opening disposed at an insulator layer posterior surface; (e) depositing a porous layer on the insulator layer posterior surface at the bore anterior opening; and (f) introducing a hole within the insulator layer in an anterior position over a substrate bore, wherein the hole comprises an anterior hole opening disposed at an insulator layer anterior surface and a hole posterior terminus disposed at the insulator layer posterior terminus. Non-limiting examples of nanowell chips that can be manufactured by such a process include those illustrated in FIG. 2D and FIG. 2E.

In alternative embodiments, a nanowell chip can be manufactured by a process that includes: (a) providing a porous substrate comprising a substrate bore and an intermediary layer disposed on an anterior surface of the substrate, the substrate bore comprising a posterior bore opening disposed at a substrate posterior surface and an anterior terminus disposed within the substrate, the intermediary layer comprising a support layer disposed on the substrate anterior surface and an active layer disposed on an anterior surface of the support layer; (b) depositing an insulator layer on an anterior surface of the intermediary layer; and (c) introducing a hole within the insulator layer, wherein the hole comprises an anterior hole opening disposed at an insulator layer anterior surface and a hole posterior terminus disposed at the insulator layer posterior surface. A non-limiting example of a nanowell chip that can be manufactured by such a process is illustrated in FIG. 2F.

In alternative embodiments, a nanowell chip can be prepared by a process that includes: (a) providing a porous substrate comprising a substrate bore and an intermediary layer disposed on an anterior surface of the substrate, the substrate bore comprising a posterior bore opening disposed at a substrate posterior surface and an anterior terminus disposed within the substrate, the intermediary layer comprising a support layer disposed on the substrate anterior surface and an active layer disposed on an anterior surface of the support layer; (b) depositing an electrode layer on an anterior surface of the intermediary layer; (c) depositing an insulator layer on an anterior surface of the electrode layer; and (d) introducing a hole within the insulator layer and within the electrode layer, wherein the hole comprises an anterior hole opening disposed at an insulator layer anterior surface and a hole posterior terminus disposed at the intermediary layer anterior surface. A non-limiting example of a nanowell chip that can be manufactured by such a process is illustrated in FIG. 2C.

An assembly can be prepared by joining a nanowell chip to an electrode chip via a partition member or partition members. A nanowell chip and an electrode chip sometimes are provided each without a joined partition member. In certain implementations, a nanowell chip joined to a partition member is provided, or an electrode chip joined to a partition member is provided, or a nanowell chip joined to a partition member and an electrode chip joined to a partition member are provided. In certain implementations, a partition member can be joined to a nanowell chip posterior surface to generate a nanowell chip/partition member assembly and a posterior surface of the partition member of the nanowell chip/partition member assembly can be joined to an anterior surface of an electrode chip. In certain implementations, a partition member can be joined to an electrode chip anterior surface to generate an electrode chip/partition member assembly and an anterior surface of the partition member of the electrode chip/partition member assembly can be joined to a posterior surface of a nanowell chip. In certain implementations, a partition member can be joined simultaneously to a posterior surface of a nanowell chip and an anterior surface of an electrode chip. In certain implementations, a first partition member can be joined to a posterior surface of a nanowell chip, a second partition member can be joined to an anterior surface of an electrode chip, and the first partition member and the second partition member can be joined (for example, a posterior surface of the first partition member can be joined to an anterior surface of the second partition member). A partition member can be joined to a nanowell chip and/or electrode chip by a suitable process, and may be joined with or without an adhesive.

An assembly or assembly component can be provided with or without fluid. For implementations in which fluid is provided, fluid can be introduced to an assembly component any suitable stage of assembly. In certain implementations, after a partition member is joined to an anterior surface of an electrode chip, fluid can be introduced to voids in the partition member, prior to, after or at the same time that an anterior surface of the partition member is joined to another member (for example, joined to a posterior surface of a nanowell chip or joined to a separate partition member that is joined to a posterior surface of a nanowell chip). In certain implementations, fluid is introduced to nanowells of a nanowell chip before, after or at the same time that a posterior surface of the nanowell chip is joined to a partition member. In certain implementations, (i) a first partition member is joined to a posterior surface of a nanowell chip and fluid is introduced to nanowells of the nanowell chip; (ii) a second partition member is joined to an anterior surface of an electrode chip and fluid is introduced to voids of the second partition member; and (iii) the first partition member and the second partition member are joined. In certain implementations, one or more portions of an assembly component are sealed after fluid is introduced. For example, an anterior surface surrounding a nanowell of a nanowell chip can be sealed with a membrane, and the membrane optionally can be removed during preparation for use of the nanowell chip in analyzing an analyte, or optionally can be left in place during preparation and/or use of the nanowell chip in analyzing an analyte.

### Use of Nanowell Chips Containing a Porous Layer

In alternative embodiments, a nanowell chip assembly is utilized in a method for analyzing an analyte. A method for analyzing an analyte can include contacting a nanowell chip assembly described herein with an analyte, where the nanowell chip includes a membrane over each nanowell on the nanowell chip and a nanopore reader in each membrane; translocating the analyte through the nanopore reader, across the membrane, and into the nanowell, where the analyte cannot transmit through the porous layer of the nanowell chip; and analyzing the analyte in the nanowell. In certain implementations, a method of analysis includes obtaining current measurements as, or after, or as and after, the analyte translocates through the nanopore reader and/or obtaining current measurements when the analyte is in the nanowell; and analyzing the analyte according to the current measurements. In certain instances the analyte is a polymer or polymer unit.

Certain implementations include determining a sequence of polymer units in a polymer in a method that includes capturing the polymer in the nanopore reader; translocating the polymer or polymer units through the nanopore reader and obtaining current measurements; and determining the sequence according to the current measurements.

In certain implementations a method for analyzing an analyte can include contacting a nanowell chip assembly described herein with an analyte, where the nanowell chip includes a membrane over each nanowell on the nanowell chip. An analyte can interact with the membrane and traverse the membrane into the nanowell (for example, for implementations in which no nanopore reader is present in the membrane). For implementations in which a membrane includes an inserted nanopore reader, an analyte can interact with the nanopore reader and translocate through the reader into the nanowell. An analyte often moves through a nanopore reader in a cis to trans direction, from bulk solution into a nanowell confined volume, and/or can move through a nanopore reader in a trans to cis direction, from within a nanowell confined volume into bulk solution. When in the nanowell, the analyte typically cannot transmit through the porous layer of the nanowell chip. A method for analyzing an analyte can include analyzing the analyte as the analyte moves through a nanopore reader in the cis direction and/or trans direction, and sometimes in the nanowell. Where the analyte is a polymer, a polymer unit can be analyzed as the polymer, or a polymer unit separated from the polymer, moves through a nanopore reader in the cis direction and/or the trans direction.

In certain implementations, a method of analysis includes obtaining current measurements as, or after, or as and after, the analyte interacts with and translocates across the membrane and/or a nanopore reader. A method of analysis often includes obtaining current measurements when the analyte is in the nanowell. A method of analysis typically includes analyzing the analyte according to the current measurements. In certain instances the analyte is a polymer or polymer unit.

In certain implementations a method of analysis includes microscopy based-imaging of the nanowell or the nanowell volume, including but not limited to fluorescence microscopy or fluorescence imaging, as, or after, or as and after, the analyte interacts with and translocates across the membrane into the confined nanowell volume.

The invention will be further described with reference to the examples described herein; however, it is to be understood that the invention is not limited to such examples.

### EXAMPLES

### Example 1: Nanowell Chip Fabrication and System Assembly

Described in this example is a process for manufacturing a nanowell chip and assembling it into a sensing system. A porous bottom nanowell chip, which is the top component of the chip stack illustrated in FIG. 6A), was fabricated by the following process The process began with patterning gold leads, contact pads, along with circular top surface metallic layer spots or pads, as shown in FIG. 6C, on the top surface of a borosilicate glass wafer. These contact pads and leads serve as the electrical connections used to detect glass wafer etching breakthrough, and as the surface for electrochemical growth of the mesoporous layers. Next, a 1-micron thick film of parylene-c was grown via chemical vapor deposition on the topside of the glass wafer over the gold structures, and nanoscale well(s) were patterned through this layer, forming nanowells, via oxygen plasma etching. This film is shown in FIG. 6C, and the well is shown in FIG. 6E. With careful alignment of the top and bottom sides of the glass wafer, 300 micron holes were developed through a protective layer of chrome/gold/photoresist on the backside of the wafer directly beneath the nanowell(s). This protect layer isolated the hydrofluoric acid etchant to dissolve hemispherical domes (FIG. 6D) through the backside of the glass wafer, until the etching solution was electrically sensed at the top surface metallic layer spots, at which point the etching was immediately quenched, forming backside etched domes and the frontside nanowells separated by a thin film of gold. Next, a mesoporous silica film was grown over the exposed gold within the backside etched dome. The film was formed using a tetraethylorthosilicate solution that grew with a homogeneous array of approximately 1.6 nm pores templated by the surfactant CTAB. The porous film condensed at the gold surface when a potential was applied between the top surface metallic layer spots and an external reference electrode, which reduced water and increased local pH. In the last fabrication step, the top metallic surface layer was etched out of the bottom of the top side nanowell(s) to provide a conductive path between the top and bottom of the wafer while leaving the rim of gold at the bottom of the nanowell sitting on top of the mesoporous silica layer (as depicted in FIG. 6A). FIG. 6F shows a brightfield image taken from the underside of the nanowell device and a darkfield image of the same region is shown in FIG. 6G that shows a light scattering silica aggregate floating on mesoporous silica that spans the nanowell. Once the nanowell chips were fabricated each was integrated into a full system by seating the chip onto a PDMS gasket with cutouts for each individual nanowell channel. They were then filled with the backside buffer solution and placed on an Ag/AgCl electrode chip, with the macroscale, large area working electrodes positioned precisely below each individual etched dome/nanowell. A Teflon cup/clamp then was compressed onto the chip/gasket stack to make electrical contact with underlying electrodes and provide the cis experimental chamber for the chip. FIG. 6B shows a photo of a functional chip installed in the center of the Teflon cup and amplifiers in a completed system.

### Example 2: Nanowell Chip System Characterization

To confirm the successful growth of a mesoporous silica membrane the nanowell chip described in Example 1 was characterized and its electrical performance was assessed at different fabrication steps. When initially setting up a test cell with 1M KCl buffer on both the trans and cis sides of the chip and the CTAB template still in place (filling the mesoporous silica porous layers), the resistance was greater than 10 GOhms. There was less than 10 pA of current with 100 mV applied. After extracting the CTAB by exchanging the buffer with acidified ethanol and then back to 1M KCl the small area porous layer had a resistance of approximately 20 MOhms. There was approximately 5 nA with 100 mV applied. For comparison, this resistance was 500 times higher than an open aperture without a mesoporous silica membrane in place. The resistance of the mesoporous silica membrane was about 50 times lower than that of a top layer membrane over the nanowell with a single ion channel, transmembrane protein or biological nanopore within it, and therefore had a minimal impact on its open channel conductance. FIG. 7A plots the current vs voltage response of the open aperture with 1M KCl for comparison with the response of the mesoporous silica before and after CTAB extraction along with the curve recorded from fully formed device with a planar lipid bilayer and a single alpha-hemolysin channel within it. FIG. 7B plots the conductance of these as a function of KCl concentration on a log-log scale. The open aperture reflected the linearity of bulk solution conductivity, and the mesoporous silica showed classic enhancement of conductivity that leveled off at low KCl concentrations due to the charged silanol groups of the silica pore walls. Fabrication of these mesoporous silica membranes was robust with electrical behaviors being reproducible over dozens of chips. Each chip was reusable multiple times and the mesoporous silica was stable against etching over time in buffered electrolyte solutions with a pH between 5 and 7.

### EXAMPLES OF EMBODIMENTS

Listed hereafter are non-limiting examples of embodiments of the technology.
A1. A nanowell chip, comprising:
   a substrate comprising a substrate posterior surface, a substrate anterior surface and a substrate bore;
   the substrate bore comprising a posterior bore opening disposed at the substrate posterior surface and an anterior bore opening disposed at the substrate anterior surface;
   an insulator layer comprising a portion in association with a portion of the substrate anterior surface;
   a porous layer in association with a portion of the insulator layer disposed at the anterior bore opening of the substrate; and
   a nanowell disposed in the insulator layer, the nanowell comprising a nanowell anterior opening disposed at an insulator layer anterior surface and a nanowell posterior terminus disposed at a porous layer anterior surface, wherein the nanowell is aligned with the anterior bore opening of the substrate.
A2. The nanowell chip of embodiment A1, wherein:
   the insulator layer is disposed on a portion of the substrate anterior surface;
   the porous layer is disposed on a portion of the insulator layer disposed at the anterior bore opening of the substrate.
A3. The nanowell chip of embodiment A1, comprising an electrode layer joined to the substrate anterior surface, the electrode layer comprising a portion disposed at the anterior bore opening, wherein:
   the insulator layer comprises a portion disposed on an electrode layer anterior surface;
   the porous layer is disposed on the electrode layer portion disposed at the anterior bore opening; and
   the nanowell is disposed in the insulator layer and in the electrode layer.
A4. A nanowell chip, comprising:
   a substrate comprising a substrate posterior surface, a substrate anterior surface and a substrate bore;
   the substrate bore comprising a posterior bore opening disposed at the substrate posterior surface and an anterior bore opening disposed at the substrate anterior surface;
   an electrode layer joined to a portion of the substrate anterior surface, the electrode layer comprising an anterior surface and a posterior surface comprising a portion disposed at the anterior bore opening;
   an insulator layer disposed on the electrode layer anterior surface and the substrate anterior surface, the insulator layer comprising an insulator layer anterior surface and an insulator layer posterior surface;
   a porous layer disposed on the electrode layer portion, the porous layer comprising a porous layer anterior surface joined to the electrode layer portion and a porous layer posterior surface disposed in the substrate bore; and a nanowell disposed in the insulator layer and in the electrode layer, the nanowell comprising a nanowell anterior opening disposed at the insulator layer anterior surface and a nanowell posterior terminus disposed at the porous layer anterior surface, wherein the nanowell is aligned with the anterior bore opening of the substrate.
A5. The nanowell chip of any one of embodiments A1-A4, comprising no electrode disposed at the nanowell posterior terminus.
A6. The nanowell chip of any one of embodiments A1-A5, wherein the porous layer comprises an array of axially aligned pores.
A7. The nanowell chip of any one of embodiments A1-A6, wherein the porous layer comprises negatively charged pores.
A8. The nanowell chip of any one of embodiments A1-A7, wherein the nanowell comprises a width and the porous layer comprises a width greater than the nanowell width.
A9. The nanowell chip of any one of embodiments A1-A8, wherein the porous layer selectively permits transmission of supporting electrolyte and does not permit transmission of an analyte.
A10. The nanowell chip of any one of embodiments A1-A9, wherein the porous layer comprises one or more of mesoporous silica, mesoporous titania, anodic aluminum oxide, a molecular thin film, graphene, graphene oxide, a polymer, polyvinyl chloride, polyetheretherketone, block copolymer, triblock copolymer, transition metal carbide, transition metal nitride, boron nitride, carbon nanotubes, molybdenum disulfide, chemically modified glass frit, sol-gel, chemically modified sol-gel, metal organic framework, and solid-state nanopores.
A11. The nanowell chip of any one of embodiments A1-A10, wherein the porous layer comprises a thickness of about 10 nanometers to about 10000 nanometers, or about 10 nanometers to about 150 nanometers, or about 50 nanometers to about 1000 nanometers, or about 750 nanometers to about 10000 nanometers.
A12. The nanowell chip of any one of embodiments A1-A11, wherein the insulator layer comprises one or more of an epoxy, polyimide, parylene, photoresist, polystyrene, fluoropolymer, silicon dioxide and silicon nitride, and optionally a bisphenol A Novolac epoxy.
A13. The nanowell chip of any one of embodiments A1-A12, wherein the insulator layer comprises a maximum thickness of about 50 nanometers to about 5000 nanometers, and/or a minimum thickness of about 50 nanometers to about 500 nanometers, or about 250 nanometers to about 1000 nanometers, or about 750 nanometers to about 5000 nanometers, or about 5000 nanometers to about 100,000 nanometers.
A14. The nanowell chip of any one of embodiments A1-A13, wherein the insulator layer comprises a width of about 50 nanometers to about 5000 nanometers, or about 50 nanometers to about 500 nanometers, or about 250 nanometers to about 1000 nanometers, or about 750 nanometers to about 5000 nanometers, or about 5000 nanometers to about 100,000 nanometers.
A15. The nanowell chip of any one of embodiments A1-A14, wherein the insulator layer comprises a hole coincident with a nanowell.
A16. The nanowell chip of any one of embodiments A3-A15, wherein the electrode layer is a metallic layer.
A17. The nanowell chip of any one of embodiments A3-A16, wherein the electrode layer comprises one or more of gold, chrome, indium tin oxide (ITO), platinum and titanium nitride.
A18. The nanowell chip of any one of embodiments A3-A17, wherein the electrode layer comprises a maximum thickness of about 5 to about 1000 nanometers, about 5 nanometers to about 100 nanometers, about 20 nanometers to about 200 nanometers, or about 100 nanometers to about 1000 nanometers, or about 1000 nanometers to about 10000 nanometers.
A19. The nanowell chip of any one of embodiments A3-A18, wherein the electrode layer comprises a hole coincident with a nanowell, and optionally coincident with a hole in the insulator layer.
A20. The nanowell chip of any one of embodiments A1-A19, wherein the substrate comprises one or more of glass, sapphire, ceramic, oxide-coated silicon, polycarbonate, polyimide, and aluminum oxide, and optionally anodic aluminum oxide or anisotropic anodic aluminum oxide.
A21. The nanowell chip of any one of embodiments A1-A20, wherein the substrate comprises a maximum thickness of about 20 to about 2000 micrometers, and optionally comprise a maximum thickness of: about 20 micrometers to about 100 micrometers, or about 75 micrometers to about 500 micrometers, or about 250 micrometers to about 25000 micrometers.
A22. The nanowell chip of any one of embodiments A1-A21, wherein the substrate bore comprises a width of about 1 micrometer to about 500 micrometers, and optionally about 1 micrometer to about 50 micrometers, or about 20 to about 250 micrometers, or about 100 micrometers to about 5000 micrometers, at the substrate anterior surface, and a width of about 1 micrometer to about 2000 micrometers, and optionally about 1 micrometer to about 50 micrometers, or about 25 micrometers to about 500 micrometers, or about 250 micrometers to about 20000 micrometers, at the substrate posterior surface.
A23. The nanowell chip of any one of embodiments A1-A22, wherein an interior surface of the substrate bore comprises one or more of: a curved interior surface, angled interior surface, a hemisphere volume, hemispheroid volume, frustrum volume, cylinder volume or cylindroid volume.
A24. The nanowell chip of any one of embodiments A1-A23, comprising a membrane disposed at the nanowell anterior opening.
A25. The nanowell chip of embodiment A11, wherein the membrane is a planar lipid bilayer (PLB).
A26. The nanowell chip of any one of embodiments A1-A25, comprising a nanopore reader disposed at the nanowell anterior opening, and optionally in a membrane.
A27. The nanowell chip of any one of embodiments A3-A26, wherein the electrode layer comprises gold.
A28. The nanowell chip of any one of embodiments A1-A27, comprising a plurality of nanowells disposed in the insulator layer, and in an optional electrode layer.
A29. The nanowell chip of embodiment A28, wherein the plurality of nanowells are disposed in an array of nanowells.
A30. The nanowell chip of any one of embodiments A1-A29, wherein one or more nanowells contain fluid.
A31. The nanowell chip of any one of embodiments A1-A30, in a system comprising a working electrode in association with a nanowell of the nanowell chip.
A32. The nanowell chip of embodiment A31, wherein the working electrode comprises a width of about 10 micrometers or greater, or about 100 micrometers or greater, or about 1,000 micrometers or greater, or about 5 micrometers to about 150 micrometers, or about 100 micrometers to about 500 micrometers, or about 250 micrometers to about 1000 micrometers, or about 10 micrometers to about 100 micrometers.
A33. The nanowell chip of embodiment A31 or A32, wherein the working electrode comprises a thickness of about 5 nanometers (nm) to about 1000 nm, or about 5 nm to about 100 nm, or about 50 nm to about 500 nm, or about 250 nm to about 1000 nm, or about 1000 nm to 10000 nm.
A34. The nanowell chip of any one of embodiments A31-A33, wherein the nanowell comprises a width of about 50 nanometers to about 5000 nanometers, or optionally: about 50 nanometers to about 250 nanometers, or about 100 nanometers to about 1000 nanometers, or about 1000 nanometers to about 5000 nanometers, or about 5000 nanometers to about 100000 nanometers, or about 500 nanometers to about 1000 nanometers, or about 1000 nanometers to about 5000 nanometers.
A34.1. The nanowell chip of any one of embodiments A31-A34, wherein a ratio between a working electrode width to an associated nanowell width is about 2 to about 200,000, or optionally is: about 2 to about 20,000, or about 2 to about 2,000, or about 2 to about 200, or about 5 to about 100, or about 10 to about 80, or about 10 to about 30, or about 55 to about 75.
A35. The nanowell chip of any one of embodiments A31-A34.1, wherein a minimum distance between the working electrode and a posterior terminus of a corresponding nanowell is about 1 micrometer to about 5000 micrometers, or about 1 micrometer to about 50 micrometers, or about 25 micrometers to about 250 micrometers, or about 500 micrometers to about 2000 micrometers.
A36. The nanowell chip of any one of embodiments A31-A35, comprising a reference electrode.
A37. The nanowell chip of embodiment A36, comprising an electrical circuit connecting the working electrode and the reference electrode.
A38. The nanowell chip of any one of embodiments A31-A37, wherein the working electrode is in bulk solution or is disposed on an electrode chip separate from the nanowell chip.
A39. The nanowell chip of any one of embodiments A36-A38, wherein the reference electrode is in bulk solution or is disposed on a electrode chip separate from the nanowell chip.
B1. An assembly, comprising a nanowell chip and two or more separated electrodes, wherein:
   the nanowell chip comprises two or more nanowells and a porous layer disposed in a posterior position at each of the two or more nanowells;
   each electrode of the two or more separated electrodes is separated by a distance from the nanowell chip, is disposed in a posterior position relative to the nanowell chip, and is in electroconductive association with one corresponding nanowell of the two or more nanowells.
B2. The assembly of embodiment B1, wherein each electrode of the two or more electrodes is a working electrode and is in electroconductive association with one nanowell of the two or more nanowells and is not in electroconductive association with another nanowell of the two or more nanowells.
B3. The assembly of embodiment B1 or B2, wherein each of the two or more nanowells has a nanowell width and the electrode in electroconductive association with the nanowell comprises an electrode width greater than the nanowell width.
B4. The assembly of any one of embodiments B1-B3, comprising a partition member disposed between the nanowell chip and each electrode, wherein the partition member fluidically separates each nanowell and corresponding separated electrode from each other nanowell and corresponding separated electrode.
B5. The assembly of embodiment B4, wherein the partition member comprises a gasket comprising a cavity associated with each nanowell and each corresponding separated electrode.
B6. The assembly of embodiment B4 or B5, wherein the partition member comprises a microfluidic chamber comprising a cavity associated with each nanowell and each corresponding separated electrode.
B7. The assembly of any one of embodiments B4-B6, wherein:
   the partition member comprises a plurality of orifices, and each of the orifices is in association with one nanowell of the two or more nanowells and one corresponding electrode of the two or more electrodes.
B8. The assembly of any one of embodiments B1-B7, wherein the one or more separated electrodes are disposed on an electrode chip.
B9. The assembly of any one of embodiments B4-B8, wherein the partition member comprises a first partition member component joined to the nanowell chip and a second partition member component joined to the electrode chip.
B10. The assembly of any one of embodiments B1-B9, wherein the nanowell chip is a nanowell chip of any one of embodiments A1-A39.
B11. The assembly of any one of embodiments B1-B10, wherein the one or more separated electrodes comprise silver.
B12. The assembly of any one of embodiments B1-B11, wherein (i) one or more nanowells of the two or more nanowells include fluid, or (ii) one or more orifices of the partition member include fluid; or (iii) a combination of (i) and (ii).
B13. The assembly of any one of embodiments B1-B12, wherein the two or more electrodes comprise two or more working electrodes.
B14. The assembly of any one of embodiments B2-B13, wherein each of the working electrodes are not in electroconductive association.
B15. The assembly of any one of embodiments B2-B14, wherein each of the working electrodes comprises a width of about 10 micrometers or greater, or optionally: about 100 micrometers or greater, or about 1,000 micrometers or greater, or about 5 micrometers to about 150 micrometers, or about 100 micrometers to about 500 micrometers, or about 250 micrometers to about 1000 micrometers, or about 10 micrometers to about 100 micrometers; and each of the nanowells comprises a width of about 50 nanometers to about 5000 nanometers, or optionally: about 50 nanometers to about 250 nanometers, or about 100 nanometers to about 1000 nanometers, or about 1000 nanometers to about 5000 nanometers, or about 5000 nanometers to about 100000 nanometers, or about 500 nanometers to about 1000 nanometers, or about 1000 nanometers to about 5000 nanometers.
B15.1. The assembly of any one of embodiments B2-B15, wherein a ratio between a working electrode width to an associated nanowell width is about 2 to about 200,000, or optionally is: about 2 to about 20,000, or about 2 to about 2,000, or about 2 to about 200, or about 5 to about 100, or about 10 to about 80, or about 10 to about 30 or about 55 to about 75.
B16. The assembly of any one of embodiments B2-B15.1, wherein a minimum distance between an anterior surface of a working electrode and a floor of a corresponding nanowell is about 1 micrometer to about 25000 micrometers, or about 1 micrometer to about 50 micrometers, or about 25 micrometers to about 250 micrometers, or about 500 micrometers to about 2000 micrometers.
B17. The assembly of any one of embodiments B1-B16, wherein the two or more electrodes comprise two more working electrodes and one or more reference electrodes.
B18. The assembly of embodiment B17, wherein the one or more reference electrodes are in bulk solution or disposed on the electrode chip.
B19. The assembly of any one of embodiments B1-B18, wherein the partition member is an insulating partition member.
B20. The assembly of any one of embodiments B1-B19, wherein the partition member comprises one or more of polydimethylsiloxane (PDMS), silicone, acrylic, polycarbonate, polytetrafluoroethylene and a fluoropolymer.
B21. The assembly of any one of embodiments B1-B20, wherein the partition member comprises a thickness of about 10 micrometers to about 1000 micrometers, or about 10 micrometers to about 100 micrometers, or about 50 micrometers to about 500 micrometers, or about 250 micrometers to about 10000 micrometers.
B22. The assembly of any one of embodiments B1-B21, wherein the electrode chip comprises a substrate, and optionally the substrate is an insulating substrate.
B23. The assembly of embodiment B22, wherein the substrate comprises one or more of glass, sapphire, ceramic, oxide-coated silicon, polycarbonate, polyimide and printed circuit board.
B24. The assembly of any one of embodiments B1-B23, wherein each electrode disposed on the electrode chip comprises a thickness of about 5 nanometers (nm) to about 1000 nm, or about 5 nm to about 100 nm, or about 50 nm to about 500 nm, or about 250 nm to about 1000 nm, or about 1000 nm to 10000 nm.
B25. The assembly of any one of embodiments B1-B24, wherein each electrode disposed on the electrode chip comprises one or more of gold, chrome, indium tin oxide (ITO), platinum, titanium, silver, and titanium nitride.
C1. A process of manufacturing a nanowell chip, comprising:
   (a) providing a substrate comprising a substrate bore and an electrode layer joined to a substrate anterior surface, the substrate bore comprising a posterior bore opening disposed at a substrate posterior surface and an anterior terminus disposed within the substrate;
   (b) optionally patterning the electrode layer whereby an electrode layer portion is disposed in an anterior position over a substrate bore;
   (c) depositing an insulator layer on an anterior surface of the electrode layer;
   (d) introducing a hole within the insulator layer in an anterior position over a substrate bore, wherein the hole comprises an anterior hole opening disposed at an insulator layer anterior surface and a hole posterior terminus disposed at the electrode layer;
   (e) deepening the substrate bore, whereby the substrate bore comprises a bore anterior opening disposed at an electrode layer posterior surface;
   (f) depositing a porous layer on the electrode layer posterior surface at the bore anterior opening; and
   (g) etching the electrode layer disposed at the hole posterior terminus, thereby forming a nanowell comprising an anterior nanowell opening disposed at an insulator layer anterior surface and a nanowell posterior terminus disposed at a porous layer anterior surface.
C2. The process of embodiment C1, comprising electrically detecting formation of the bore anterior opening at the electrode layer posterior surface.
C3. The process of embodiment C1 or C2, wherein the nanowell chip is a nanowell chip of any one of embodiments A1-A39.
C4. The process of any one of embodiments C1-C3, comprising etching the substrate bore prior to (a).
C5. The process of embodiment C4, wherein the etching is stopped before breakthrough of the bore at a substrate anterior surface.
C6. The process of embodiment C4 or C5, wherein the etching comprises contacting the substrate with hydrofluoric acid, potassium hydroxide, and/or phosphoric acid or comprises reactive ion etching.
C7. The process of any one of embodiments C4-C6, comprising applying a protective layer on a substrate posterior surface and/or substrate anterior surface prior to the etching, and removing the protective layer after the etching.
C8. The process of any one of embodiments C1-C7, comprising applying the electrode layer to the substrate prior to (a).
C9. The process of embodiment C8, wherein the electrode layer is applied after the etching of any one of embodiments C4-C7.
C10. The process of embodiment C8 or C9, wherein applying the electrode layer comprises one or more of sputtering, chemical vapor deposition electron beam evaporation.
C11. The process of any one of embodiments C1-C10, wherein optional part (b) comprises patterning the electrode layer by a lithographic process.
C12. The process of embodiment C11, wherein the lithographic process includes one or more of a spin coat photoresist, develop photoresist, and etch metals or liftoff resist.
C13. The process of any one of embodiments C1-C12, wherein in part (c) the insulator film is grown, deposited, spin coated, 3D printed, or applied to the top surface.
C14. The process of any one of embodiments C1-C13, wherein (d) comprises a photo lithographic process, electron beam lithographic process and/or plasma/reactive ion etching process.
C15. The process of any one of embodiments C1-C14, wherein (e) comprises etching the substrate bore.
C16. The process of embodiment C15, wherein the etching comprises contacting the bore with hydrofluoric acid, potassium hydroxide, and/or phosphoric acid or comprises reactive ion etching.
C17. The process of embodiment C15 or C16, wherein the etching is stopped (i) after a predetermined amount of time, and/or (ii) after detecting breakthrough of the bore at the electrode layer by electrically monitoring the electrode layer.
C18. The process of any one of embodiments C1-C17, wherein (f) comprises a template assisted process.
C19. The process of embodiment C18, wherein (f) comprises depositing a structure-directing material and a guest material on the electrode layer.
C20. The process of embodiment C19, wherein the structure-directing material comprises a surfactant or block copolymer and the guest material comprises silica or titania sol gel.
C21. The process of embodiment C19 or C20, comprising removing the structure-directing material and/or a guest material.
C22. The process of any one of embodiments C1-C21, wherein (f) comprises an electrochemical process.
C23. The process of embodiment C22, wherein the electrochemical process comprises aluminum anodization followed by etching with phosphoric acid to generate anodic aluminum oxide; water reduction to change pH locally and drive condensation of sol gel based films, which optionally comprises mesoporous silica or titania; co-deposition of a metal, which optionally is nickel, gold and/or silver); and/or deposition of a polymers, which optionally comprises polyaniline polymer (PANI polymer), poly(3,4-ethylenedioxythiophene) polymer (PEDOT polymer), polypyrrole polymer (PPy polymer)), optionally with a porogen that is subsequently removed.
C24. The process of any one of embodiments C1-C23, wherein (f) comprises a chemical vapor deposition process.
C25. The process of embodiment C24, wherein the chemical vapor deposition process comprises co-deposition of a monomers, optionally a methacrylate, and an initiator, optionally a peroxide, and a porogen, optionally a siloxane and/or naphthalene.
C26. The process of any one of embodiments C1-C25, wherein (f) comprises a self assembly process.
C27. The process of embodiment C26, wherein the self assembly process comprises random dense packing of nanoparticles, evaporation induced film formation via dip coating, spray coating, spin coating, or printing of precursors directly onto a surface.
C28. The process of any one of embodiments C1-C27, comprising introducing pores into the porous layer.
C29. The process of embodiment C28, comprising introducing pores by TEM drilling, dielectric breakdown, and/or track etching.
C30. The process of any one of embodiments C1-C29, wherein (g) comprises contacting the electrode layer anterior surface in an insulator layer hole with an etchant.
C31. The process of embodiment C30, wherein the etchant comprises iodine/iodide or cyanide/oxidizer) on the top surface.
C32. The process of any one of embodiments C1-C31, wherein the nanowell chip is contacted with a template or porogen, and the template or porogen is removed after (g).
C33. A process of manufacturing a nanowell chip, comprising:
   (a) providing a substrate comprising a substrate bore, the substrate bore comprising a posterior bore opening disposed at a substrate posterior surface and an anterior terminus disposed within the substrate;
   (b) depositing an insulator layer on an anterior surface of the substrate;
   (c) deepening the substrate bore, whereby the substrate bore comprises a bore anterior opening disposed at an insulator layer posterior surface;
   (d) depositing a porous layer on the insulator layer posterior surface at the bore anterior opening; and
   (e) introducing a hole within the insulator layer in an anterior position over a substrate bore, wherein the hole comprises an anterior hole opening disposed at an insulator layer anterior surface and a hole posterior terminus disposed at the insulator layer posterior terminus.
C34. The process of embodiment C33 or C34, wherein the porous layer is grown by vapor deposition or by dip coating.
C35. A process of manufacturing a nanowell chip, comprising:
   (a) providing a porous substrate and an intermediary layer disposed on an anterior surface of the substrate, the intermediary layer comprising a support layer disposed on the substrate anterior surface and an active layer disposed on an anterior surface of the support layer;
   (b) depositing an insulator layer on an anterior surface of the intermediary layer; and
   (c) introducing a hole within the insulator layer, wherein the hole comprises an anterior hole opening disposed at an insulator layer anterior surface and a hole posterior terminus disposed at the insulator layer posterior surface.
C36. A process of manufacturing a nanowell chip, comprising:
   (a) providing a porous substrate comprising a substrate bore, an intermediary layer disposed on an anterior surface of the substrate and an electrode layer disposed on an anterior surface of the intermediary layer, the intermediary layer comprising a support layer disposed on the substrate anterior surface and an active layer disposed on an anterior surface of the support layer;
   (b) depositing an insulator layer on an anterior surface of the electrode layer; and
   (c) introducing a hole within the insulator layer and within the electrode layer, wherein the hole comprises an anterior hole opening disposed at an insulator layer anterior surface and a hole posterior terminus disposed at the intermediary layer anterior surface.
C37. The process of any one of embodiments C33-C36, wherein the insulator layer is grown, deposited, spin coated, 3D printed, or applied to the anterior surface of the substrate.
C38. The process of any one of embodiments C33-C37, wherein the hole is introduced by an isotropic process, which optionally is an acid etching process, or by an anisotropic process, which optionally is a base etching process or laser process.
D1. A process of manufacturing an assembly, comprising:
   providing a nanowell chip, an electrode chip and a partition member;
   joining a partition member anterior surface to a nanowell chip posterior surface; and
   joining a partition member posterior surface to an electrode chip anterior surface.
D2. The process of embodiment D1, wherein the assembly is an assembly of any one of embodiments B1-B25 and/or the nanowell chip is a nanowell chip of any of embodiments A1-A39.
D3. The process of embodiment D1 or D2, wherein the nanowell chip is manufactured by a process of any one of embodiments C1-C38.
E1. A method for analyzing an analyte, comprising:
   contacting a nanowell chip of any one of embodiments A1-A39 or an assembly of any one of embodiments B1-B25 with an analyte, wherein the nanowell chip comprises a nanopore reader;
   translocating the analyte through the nanopore reader and into the nanowell, wherein the analyte cannot transmit through the porous layer; and
   analyzing the analyte in the nanowell.
E2. The method of embodiment E1, comprising:
   obtaining current measurements as, or after, or as and after, the analyte translocates through the nanopore reader and/or obtaining current measurements when the analyte is in the nanowell; and
   analyzing the analyte according to the current measurements.
E3. The method of embodiment E1 or E2, wherein the analyte is a polymer or polymer unit.
E4. The method of embodiment E3, wherein the analyte is a polymer.
E5. The method of embodiment E4, comprising:
   capturing the polymer in the nanopore reader;
   translocating the polymer through the nanopore reader and obtaining current measurements; and
   determining a sequence of polymer units in the polymer according to the current measurements.
F1. An article of manufacture comprising a nanowell chip of any one of embodiments A1-A39, an assembly of any one of embodiments B1-B25, a nanowell chip manufactured by a process of any one of embodiments C1-C38, and/or an assembly manufactured by a process of any one of embodiments D1-D3.

Any of the above aspects and embodiments can be combined with any other aspect or embodiment as disclosed here in the Summary, Drawings and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

Unless specifically stated or obvious from context, as used herein, the term “exemplary” means a non-limiting embodiment, feature or article.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

A number of embodiments of the invention have been described. Nevertheless, it can be understood that various modifications may be made without departing from the spirit and scope of the invention. Accordingly, other embodiments are within the scope of the following claims.

1: A nanowell chip, comprising:
a substrate comprising a substrate posterior surface, a substrate anterior surface and a substrate bore;
the substrate bore comprising a posterior bore opening disposed at the substrate posterior surface and an anterior bore opening disposed at the substrate anterior surface;
an insulator layer comprising a portion in association with a portion of the substrate anterior surface;
a porous layer in association with a portion of the insulator layer disposed at the anterior bore opening of the substrate; and
a nanowell disposed in the insulator layer, the nanowell comprising a nanowell anterior opening disposed at an insulator layer anterior surface and a nanowell posterior terminus disposed at a porous layer anterior surface, wherein the nanowell is aligned with the anterior bore opening of the substrate.

2: The nanowell chip of claim 1, wherein:
the insulator layer is disposed on a portion of the substrate anterior surface;
the porous layer is disposed on a portion of the insulator layer disposed at the anterior bore opening of the substrate.

3: The nanowell chip of claim 1, comprising an electrode layer joined to the substrate anterior surface, the electrode layer comprising a portion disposed at the anterior bore opening, wherein:
the insulator layer comprises a portion disposed on an electrode layer anterior surface;
the porous layer is disposed on the electrode layer portion disposed at the anterior bore opening; and
the nanowell is disposed in the insulator layer and in the electrode layer.

4: A nanowell chip, comprising:
a substrate comprising a substrate posterior surface, a substrate anterior surface and a substrate bore;
the substrate bore comprising a posterior bore opening disposed at the substrate posterior surface and an anterior bore opening disposed at the substrate anterior surface;
an electrode layer joined to a portion of the substrate anterior surface, the electrode layer comprising an anterior surface and a posterior surface comprising a portion disposed at the anterior bore opening;
an insulator layer disposed on the electrode layer anterior surface and the substrate anterior surface, the insulator layer comprising an insulator layer anterior surface and an insulator layer posterior surface;
a porous layer disposed on the electrode layer portion, the porous layer comprising a porous layer anterior surface joined to the electrode layer portion and a porous layer posterior surface disposed in the substrate bore; and
a nanowell disposed in the insulator layer and in the electrode layer, the nanowell comprising a nanowell anterior opening disposed at the insulator layer anterior surface and a nanowell posterior terminus disposed at the porous layer anterior surface, wherein the nanowell is aligned with the anterior bore opening of the substrate.

5: The nanowell chip of claim 1, comprising no electrode disposed at the nanowell posterior terminus.

6: The nanowell chip of claim 1, wherein the porous layer comprises an array of axially aligned pores, and optionally comprises negatively charged pores.

7: The nanowell chip of claim 1, wherein the porous layer selectively permits transmission of supporting electrolyte and does not permit transmission of an analyte.

8: The nanowell chip of claim 1, wherein the porous layer comprises one or more of mesoporous silica, mesoporous titania, anodic aluminum oxide, a molecular thin film, graphene, graphene oxide, a polymer, polyvinyl chloride, polyetheretherketone, block copolymer, triblock copolymer, transition metal carbide, transition metal nitride, boron nitride, carbon nanotubes, molybdenum disulfide, chemically modified glass frit, sol-gel, chemically modified sol-gel, metal organic framework, and solid-state nanopores.

9: The nanowell chip of claim 1, wherein the insulator layer comprises a hole coincident with a nanowell, and optionally: the electrode layer comprises a hole coincident with a nanowell, and/or a hole in the electrode layer is coincident with a hole in the insulator layer.

10: The nanowell chip of claim 3, wherein the electrode layer is a metallic layer.

11: The nanowell chip of claim 1, comprising a membrane disposed at the nanowell anterior opening, and optionally the membrane is a planar lipid bilayer (PLB).

12: The nanowell chip of claim 1, comprising a nanopore reader disposed at the nanowell anterior opening, and optionally in a membrane.

13: The nanowell chip of claim 1, comprising a plurality of nanowells disposed in the insulator layer, and in an optional electrode layer, and optionally the plurality of nanowells are disposed in an array of nanowells.

14: The nanowell chip of claim 1, wherein one or more nanowells contain fluid.

15: The nanowell chip of claim 1, in a system comprising a working electrode in association with a nanowell of the nanowell chip, and optionally: wherein the working electrode is in bulk solution or is disposed on an electrode chip separate from the nanowell chip, and optionally the working electrode comprises a width of about 10 micrometers or greater, or about 100 micrometers or greater, or about 1,000 micrometers or greater, or about 5 micrometers to about 150 micrometers, or about 100 micrometers to about 500 micrometers, or about 250 micrometers to about 1000 micrometers, or about 10 micrometers to about 100 micrometers; and optionally, wherein the nanowell comprises a width of about 50 nanometers to about 5000 nanometers, or optionally: about 50 nanometers to about 250 nanometers, or about 100 nanometers to about 1000 nanometers, or about 1000 nanometers to about 5000 nanometers, or about 5000 nanometers to about 100000 nanometers, or about 500 nanometers to about 1000 nanometers, or about 1000 nanometers to about 5000 nanometers.

16. (canceled)

17: The nanowell chip of claim 15, wherein:
(a) a minimum distance between an anterior surface of the working electrode and a posterior terminus of a corresponding nanowell is about 1 micrometer to about 25000 micrometers, or about 1 micrometer to about 50 micrometers, or about 25 micrometers to about 250 micrometers, or about 500 micrometers to about 2000 micrometers;
(b) comprising a reference electrode, and optionally: wherein the reference electrode is in bulk solution or is disposed on a electrode chip separate from the nanowell chip; or
(c) comprising an electrical circuit connecting the working electrode and the reference electrode.

18-19. (canceled)

20: An assembly comprising a nanowell chip and two or more separated electrodes, wherein:
the nanowell chip comprises two or more nanowells and a porous layer disposed in a posterior position at each of the two or more nanowells;
each electrode of the two or more separated electrodes is separated by a distance from the nanowell chip, is disposed in a posterior position relative to the nanowell chip, and is in electroconductive association with one corresponding nanowell of the two or more nanowells.

21: The assembly of claim 20, wherein:
(a) each electrode of the two or more electrodes is a working electrode and is in electroconductive association with one nanowell of the two or more nanowells and is not in electroconductive association with another nanowell of the two or more nanowells;
(b) each of the two or more nanowells has a nanowell width and the electrode in electroconductive association with the nanowell comprises an electrode width greater than the nanowell width;
(c) comprising a partition member disposed between the nanowell chip and each electrode, wherein the partition member fluidically separates each nanowell and corresponding separated electrode from each other nanowell and corresponding separated electrode,
and optionally the partition member comprises a gasket comprising a cavity associated with each nanowell and each corresponding separated electrode;
and optionally the partition member comprises a microfluidic chamber comprising a cavity associated with each nanowell and each corresponding separated electrode;
and optionally the partition member comprises a plurality of orifices, and each of the orifices is in association with one nanowell of the two or more nanowells and one corresponding electrode of the two or more electrodes;
(d) the one or more separated electrodes are disposed on an electrode chip;
(e) the partition member comprises a first partition member component joined to the nanowell chip and a second partition member component joined to the electrode chip;
(f) the nanowell chip is a nanowell chip as set forth in claim 1;
(g) each of the working electrodes comprises a width of about 10 micrometers or greater, or about 100 micrometers or greater, or about 1,000 micrometers or greater, or about 5 micrometers to about 150 micrometers, or about 100 micrometers to about 500 micrometers, or about 250 micrometers to about 1000 micrometers, or about 10 micrometers to about 100 micrometers; and optionally, each of the nanowells comprises a width of about 50 nanometers to about 5000 nanometers, or optionally: about 50 nanometers to about 250 nanometers, or about 100 nanometers to about 1000 nanometers, or about 1000 nanometers to about 5000 nanometers, or about 5000 nanometers to about 100000 nanometers, or about 500 nanometers to about 1000 nanometers, or about 1000 nanometers to about 5000 nanometers;
(h) a minimum distance between an anterior surface of a working electrode and a floor of a corresponding nanowell is about 1 micrometer to about 25000 micrometers, or about 1 micrometer to about 50 micrometers, or about 25 micrometers to about 250 micrometers, or about 500 micrometers to about 2000 micrometers; or
(i) any combination of (a) to (h).

22-31. (canceled)

32: A process of manufacturing a nanowell chip, comprising:
(a) providing a substrate comprising a substrate bore and an electrode layer joined to a substrate anterior surface, the substrate bore comprising a posterior bore opening disposed at a substrate posterior surface and an anterior terminus disposed within the substrate;
(b) optionally patterning the electrode layer whereby an electrode layer portion is disposed in an anterior position over a substrate bore;
(c) depositing an insulator layer on an anterior surface of the electrode layer;
(d) introducing a hole within the insulator layer in an anterior position over a substrate bore, wherein the hole comprises an anterior hole opening disposed at an insulator layer anterior surface and a hole posterior terminus disposed at the electrode layer;
(e) deepening the substrate bore, whereby the substrate bore comprises a bore anterior opening disposed at an electrode layer posterior surface;
(f) depositing a porous layer on the electrode layer posterior surface at the bore anterior opening; and
(g) etching the electrode layer disposed at the hole posterior terminus, thereby forming a nanowell comprising an anterior nanowell opening disposed at an insulator layer anterior surface and a nanowell posterior terminus disposed at a porous layer anterior surface.

33: The process of claim 32, comprising electrically detecting formation of the bore anterior opening at the electrode layer posterior surface.

34. (canceled)

35: A process of manufacturing an assembly, comprising:
providing a nanowell chip, an electrode chip and a partition member;
joining a partition member anterior surface to a nanowell chip posterior surface; and
joining a partition member posterior surface to an electrode chip anterior surface.

36. (canceled)

37: A method for analyzing an analyte, comprising:
contacting a nanowell chip of claim 1 with an analyte, wherein the nanowell chip comprises a nanopore reader;
translocating the analyte through the nanopore reader and into the nanowell, wherein the analyte cannot transmit through the porous layer; and
analyzing the analyte in the nanowell.

38: The method of claim 37, wherein
(a) the method comprises: obtaining current measurements as, or after, or as and after, the analyte translocates through the nanopore reader and/or obtaining current measurements when the analyte is in the nanowell; and analyzing the analyte according to the current measurements;
(b) the analyte is a polymer or polymer unit; or
(c) the method comprises: capturing the polymer in the nanopore reader; translocating the polymer through the nanopore reader and obtaining current measurements; and, determining a sequence of polymer units in the polymer according to the current measurements.

39-40. (canceled)
